(19) United States
(12) Patent Application Publication
(10) Pub. No.: US 2026/0275408 A1
(43) Pub. Date: Sep. 17, 2026

(54) METHODS AND COMPOSITIONS FOR AMPLIFICATION AND SEQUENCING OF GENOME AND EPIGENOME

(71) Applicant: Board of Regents, The University of Texas System, Austin, TX (US)

(72) Inventors: Kaile Wang, Houston, TX (US); Nicholas Navin, Houston, TX (US); Yun Yan, Houston, TX (US); Heba Elgamal, Houston, TX (US); Yiyun Lin, Houston, TX (US); Junke Wang, Houston, TX (US)

(73) Assignee: Board of Regents, The University of Texas System, Austin, TX (US)

(21) Appl. No.: 19/162,786

(22) PCT Filed: Mar. 6, 2024
(86) PCT No.: PCT/US2024/018634

§ 371 Date: Sep. 5, 2025

Related U.S. Application Data
(60) Provisional application No. 63/488,897, filed on Mar. 7, 2023.

(51) Int. Cl. C12Q 1/6844; C12Q 1/48; C12Q 1/6806; C12Q 1/6813; C12Q 1/6869; C12Q 1/6876; C40B 40/06; C40B 50/06; C40B 70/00

(52) U.S. Cl. CPC C12Q 1/6844; C12Q 1/48; C12Q 1/6806; C12Q 1/6813; C12Q 1/6869; C12Q 1/6876; C12Y 207/07; C40B 40/06; C40B 50/06; C40B 70/00; C12Q 2600/112; C12Q 2600/156

(57) ABSTRACT

The present disclosure relates to methods and compositions for the approximately simultaneous amplification of closed chromatin region DNA and open chromatin region DNA. The present disclosure further provides methods of producing a DNA library for analysis of open chromatin region DNA and closed chromatin region DNA approximately simultaneously and methods of identifying a DNA sequence variation or an epigenetic modification in a sample. Aspects of the present disclosure further relate to a DNA library produced by the methods of the present disclosure.

### CROSS-REFERENCE TO RELATED APPLICATIONS

This application claims the priority of U.S. Provisional Appl. Ser. No. 63/488,897, filed Mar. 7, 2023, the entire disclosure of which is incorporated herein by reference.

### INCORPORATION OF SEQUENCE LISTING

A sequence listing containing the file named “MDCC002WO_ST26” which is 12.8 kilobytes (measured in MS-Windows®) and created on Mar. 4, 2024, and comprises 13 sequences, is incorporated herein by reference in its entirety.

### FIELD OF THE INVENTION

This present disclosure relates to the field of producing a DNA sequencing library for the sequencing and analysis of open chromatin region DNA and closed chromatin region DNA approximately simultaneously, and more specifically to methods of producing a DNA sequencing library that allows for the approximately simultaneous sequencing and analysis of open chromatin region DNA and closed chromatin region DNA from a small number of cells or a single cell.

### BACKGROUND OF THE INVENTION

How genetic variation impacts the epigenetic landscape and cell phenotype is one of the most fundamental questions in molecular biology. Nucleosome position in a genome has a significant regulatory function. In particular, nucleosome position modifies the in vivo availability of transcription factor binding sites as well as binding sites for the general transcription machinery, and thus has a significant effect on DNA-dependent processes such as transcription, DNA repair, replication, and recombination. Importantly, nucleosome position also affects the genomic instability in healthy and diseased tissues, such as cancer. Methods for sequencing the genome, which includes both open chromatin region DNA and closed chromatin region DNA, and the epigenome, which includes open chromatin region DNA, have emerged as powerful tools to study biological systems at a genome-wide scale. Such methods include, but are not limited to, DNA-seq, ATAC-seq, and CUT&Tag (Mehrmohamad, et al., Front. Cell Dev. Biol. 9:714687, 2021; Klemm, et al., Nature Reviews Genetics 20:207-220, 2019). Bulk sequencing methods, however, are limited to profiling a large population of cells, and thus only provide an average measurement over the entire population. This masks the variation present in complex tissues. These limitations impair the ability to determine how genetic mutations and variations impact the epigenetic landscape. To date, no methods exist which allow for the sequencing of both the closed chromatin region DNA and the open chromatin region DNA approximately simultaneously from the same low-input starting material, such as a single cell.

Although, some computational tools have been developed to infer DNA copy number information from ATAC sequencing (Nikolic, et al., Science Advances 7(42):eabg6045, 2021; Wu, et al., Nature Biotechnology 39:1259-1269, 2021), the majority of genome regions, which may contain important mutations, indels, copy number variations, and structure variations, cannot be profiled due to the limited amount of open chromatin region DNA and the biased signal of single cell ATAC-seq. Recently, a method known as scGET-seq (Tedesco, et al., Nature Biotechnology 40:235-244, 2022) was developed, which attempts to profile chromatin velocity by profiling heterochromatin and euchromatin DNA. Due, however, to limited resolution, scGET-seq could not accurately distinguish tumor cell subclones. Additionally, scGET-seq could not be used to accurately detect DNA mutations and indels.

Therefore, there remains a significant need in the art for a high resolution, comprehensive, flexible, and highly scalable approach to characterize how genetic variation impacts the epigenetic landscape within complex tissues such as tumors, which are often composed of many genetically distinct tumor subclones. In particular, methods for detecting how genetic variation affects features such as chromatin accessibility, DNA and protein interaction, and histone modifications are needed in the art. The present disclosure provides such methods which can amplify the open chromatin DNA regions and closed chromatin DNA regions from the same low input material. In particular, the methods provided herein allow for the amplification of open chromatin DNA regions and closed chromatin DNA regions from single cells, or from tens, hundreds, or thousands of cells approximately simultaneously without physically separating the two molecular pools of open chromatin region DNA and closed chromatin region DNA prior to amplification. In summary, the present disclosure provides methods wherein the open chromatin region DNA from the input material is first labeled with one set of DNA oligonucleotides or barcode, then, the chromatin of labeled input material is removed to expose the closed chromatin region DNA. The input material may be, for example, a single cell or nuclei, a cell suspension, or a cell lysate. The exposed closed chromatin DNA is then labeled with another set of DNA oligonucleotides or barcodes. A cell or sample set of oligonucleotides or barcodes can be introduced during or after adding the aforementioned two sets of oligonucleotides or barcodes. The two amplified modalities, the open chromatin region DNA and closed chromatin region DNA may then be separated by enrichment PCR. In some embodiments, the two amplified modalities may be separated by amplifying each modality using modality specific labels. Next, in certain embodiments, the two different DNA libraries may be sequenced using high-throughput DNA sequencing. The methods described herein are compatible with tube-based reactions, such as those performed in a single tube or those preformed in 8-strip tubes reaction, plate-based reactions, such as those performed in a 96-well or 384-well plate, and high density platforms, such as those performed in nanowells or nanodroplets. The amplified closed chromatin region DNA and open chromatin region DNA may be used, for example, to detect genome-wide copy number variations, DNA mutations, structural variations, and other genomic aberrations, while the amplified open chromatin region DNA may be used, for example, to detect chromatin accessibility, transcriptional regulation, epigenetic modifications, histone modifications, and cell phenotype. Moreover, since information regarding the closed chromatin region DNA and the open chromatin region DNA are measured from the same low-input material or single cell, the methods provided herein allow for the study of how DNA aberrations impact transcriptional regulation, and how these two layers of molecular information interact and affect one another. In summary, the methods of the present disclosure can be used to determine how genomic information affects epigenomic phenotype in low-input materials, including single cells. The methods provided by the present disclosure will have broad applications in the study of genome and transcriptome interactions, allowing for the study of how mutations or copy number variations affect the transcriptional regulation in normal or tumor cells, and allowing for the quantification of these effects in different cell types. The methods described herein can be used in many research areas to study the basic biology of development, tumorigenesis, and cancer progression, to identify novel predictive and prognostic biomarkers, and to identify novel drug targets in the clinic.

### SUMMARY OF THE INVENTION

In one aspect the present disclosure provides, a method of producing a DNA library for analysis of open chromatin region DNA and closed chromatin region DNA approximately simultaneously, the method comprising: a) obtaining a sample comprising chromosomal DNA; b) performing a first labeling reaction on the chromosomal DNA to label at least a first portion of open chromatin region DNA comprised within the chromosomal DNA with a first label; c) disrupting the structure of the chromosomal DNA to expose at least a first portion of closed chromatin region DNA comprised within the chromosomal DNA; d) performing a second labeling reaction on the chromosomal DNA to label: i) at least the first portion of the exposed closed chromatin region DNA with a second label; and ii) at least the first portion, or a second portion, of open chromatin region DNA with the second label; and e) performing an amplification reaction to amplify the first labeled open chromatin region DNA, the second labeled exposed closed chromatin region DNA, and the second labeled open chromatin region DNA comprised within the chromosomal DNA. In one embodiment, the sample comprises chromosomal DNA obtained from about 1 cell to about 1,000,000,000 cells. In another embodiment, the sample comprises chromosomal DNA obtained from a single cell. In yet another embodiment, the amplification reaction amplifies the first labeled open chromatin region DNA, the second labeled exposed closed chromatin region DNA, and the second labeled open chromatin region DNA approximately simultaneously. The first labeling reaction or the second labeling reaction, in certain embodiments, is performed by an insertional enzyme complex. In one embodiment, the insertional enzyme complex comprises a transposase. Non limiting examples of a transposase may include a Tn transposase and MuA transposase. In another embodiment, the method may further comprise performing a third labeling reaction on the chromosomal DNA to label the chromosomal DNA of the sample with a third label. The third labeling reaction and the first labeling reaction, in yet another embodiment, are performed approximately simultaneously. The third labeling reaction and the second labeling reaction, in still yet another embodiment, are performed approximately simultaneously. The third labeling reaction, in one embodiment, is performed before or after the first labeling reaction. The third labeling reaction, in another embodiment, is performed before or after the second labeling reaction. The third labeling reaction and the amplification reaction, in yet another embodiment, are performed approximately simultaneously. In still yet another embodiment, the first label, the second label, or the third label is a nucleotide barcode label. In one embodiment, the first label and the second label are different. In another embodiment, the first label and the third label are different. In yet another embodiment, the second label and the third label are different.

In certain embodiments, the methods of the present disclosure may further comprise detecting at least one DNA sequence variation in the amplified second labeled exposed closed chromatin region DNA. In one embodiment, the at least one DNA sequence variation is selected from the group consisting of a mutation, a copy number alteration, a structural variation, and a DNA modification. In another embodiment, the methods of the present disclosure may further comprise performing an Assay for Transposase-Accessible Chromatin (ATAC) analysis on the open chromatin region DNA to measure chromatin accessibility differences in the chromosomal DNA. In yet another embodiment, the methods of the present disclosure may further comprise performing a Cleavage Under Targets and Tagmentation (Cut&Tag) analysis of the open chromatin region DNA to measure protein:DNA interactions of the chromosomal DNA. In still yet another embodiment, the methods of the present disclosure may comprise detecting at least one epigenetic modification in close proximity to the open chromatin region DNA.

In another aspect, the sample comprises a plurality of cells, and the method further comprises separating the sample into a plurality of sub-samples, each comprising a single cell. In one embodiment, the separating is performed prior to disrupting the structure of the chromosomal DNA. In another embodiment, the method further comprises performing a third labeling reaction on the chromosomal DNA to label the chromosomal DNA of at least one sub-sample with a third label. In yet another embodiment, the method further comprises performing a third labeling reaction on the chromosomal DNA to label the chromosomal DNA of each sub-sample with a unique third label.

In yet another aspect the methods provided by the present disclosure may further comprise separating the first labeled open chromatin region DNA from the second labeled exposed closed chromatin region DNA and the second labeled open chromatin region DNA following the step of performing an amplification reaction to amplify the first labeled open chromatin region DNA, the second labeled exposed closed chromatin region DNA, and the second labeled open chromatin region DNA comprised within the chromosomal DNA. In one embodiment, the methods provided by the present disclosure may further comprise performing a DNA sequencing reaction to obtain the DNA sequence of the first labeled open chromatin region DNA, the second labeled exposed closed chromatin region DNA, or the second labeled open chromatin region DNA. In yet another aspect, the present disclosure provides a DNA library produced by the methods described herein.

In still yet another aspect the present disclosure provides a method of producing a DNA library for analysis of open chromatin region DNA and closed chromatin region DNA approximately simultaneously, the method comprising: a) obtaining a plurality of samples comprising chromosomal DNA; b) performing a first labeling reaction on the chromosomal DNA of each sample to label at least a first portion of open chromatin region DNA comprised within the chromosomal DNA with a first label; c) disrupting the structure of the chromosomal DNA of each sample to expose at least a first portion of closed chromatin region DNA comprised within the chromosomal DNA; d) performing a second labeling reaction to label: i) at least the first portion of the exposed closed chromatin region DNA with a second label; and ii) at least the first portion, or a second portion, of open chromatin region DNA with the second label; e) performing a third labeling reaction on the chromosomal DNA of each sample to label the chromosomal DNA of the sample with a unique third label; f) combining the first labeled open chromatin region DNA, the second labeled exposed closed chromatin region DNA, and the second labeled open chromatin region DNA from each sample of the plurality of samples; and g) performing an amplification reaction to amplify the first labeled open chromatin region DNA, the second labeled exposed closed chromatin region DNA, and the second labeled open chromatin region DNA comprised within the chromosomal DNA. In one embodiment, each sample of the plurality of samples comprises chromosomal DNA obtained from about 1 cell to about 1,000,000,000 cells. In another embodiment, each sample of the plurality of samples comprises chromosomal DNA obtained from a single cell. In yet another embodiment, the amplification reaction amplifies the first labeled open chromatin region DNA, the second labeled exposed closed chromatin region DNA, and the second labeled open chromatin region DNA approximately simultaneously. The third labeling reaction and the first labeling reaction, in one embodiment, are performed approximately simultaneously. The third labeling reaction and the second labeling reaction, in another embodiment, are performed approximately simultaneously. The third labeling reaction, in yet another embodiment, is performed before or after the first labeling reaction. The third labeling reaction, in still yet another embodiment, is performed before or after the second labeling reaction. The third labeling reaction and the amplification reaction, in one embodiment, are performed approximately simultaneously.

In one aspect, at least one sample of the plurality of samples comprises a plurality of cells, and the method further comprises separating the at least one sample into a plurality of sub-samples, each comprising a single cell. In one embodiment, the separating is performed prior to disrupting the structure of the chromosomal DNA. In another embodiment, the method further comprises separating the first labeled open chromatin region DNA from the second labeled exposed closed chromatin region DNA and the second labeled open chromatin region DNA following the step of performing an amplification reaction to amplify the first labeled open chromatin region DNA, the second labeled exposed closed chromatin region DNA, and the second labeled open chromatin region DNA comprised within the chromosomal DNA. In yet another embodiment, the method further comprises performing a DNA sequencing reaction to obtain the DNA sequence of the first labeled open chromatin region DNA, the second labeled exposed closed chromatin region DNA, or the second labeled open chromatin region DNA following the step of performing an amplification reaction to amplify the first labeled open chromatin region DNA, the second labeled exposed closed chromatin region DNA, and the second labeled open chromatin region DNA comprised within the chromosomal DNA. In another aspect, the present disclosure provides a DNA library produced by the methods descripted herein.

In another aspect, the present disclosure provides a method of identifying a DNA sequence variation or an epigenetic modification in a sample, the method comprising: a) obtaining a sample comprising chromosomal DNA; b) performing a first labeling reaction on the chromosomal DNA to label at least a first portion of open chromatin region DNA comprised within the chromosomal DNA with a first label; c) disrupting the structure of the chromosomal DNA to expose at least a first portion of closed chromatin region DNA comprised within the chromosomal DNA; d) performing a second labeling reaction to label: i) at least the first portion of the exposed closed chromatin region DNA with a second label; and ii) at least the first portion, or a second portion, of open chromatin region DNA with the second label; e) performing an amplification reaction to amplify the first labeled open chromatin region DNA, the second labeled exposed closed chromatin region DNA, and the second labeled open chromatin region DNA comprised within the chromosomal DNA; f) performing a DNA sequencing reaction to obtain the DNA sequence of the first labeled open chromatin region DNA, the second labeled exposed closed chromatin region DNA, or the second labeled open chromatin region DNA comprised within the chromosomal DNA; and g) identifying the DNA sequence variation or the epigenetic modification in the sample. In one embodiment, the sample comprises chromosomal DNA obtained from about 1 cell to about 1,000,000,000 cells. In another embodiment, the sample comprises chromosomal DNA obtained from a single cell. In yet another embodiment, the sample comprises a plurality of cells, and the method further comprises separating the sample into a plurality of sub-samples, each comprising a single cell. In still yet another embodiment, the separating is performed prior to disrupting the structure of the chromosomal DNA. In one embodiment, the amplification reaction amplifies the first labeled open chromatin region DNA, the second labeled exposed closed chromatin region DNA, and the second labeled open chromatin region DNA approximately simultaneously. In another embodiment, the method further comprises separating the first labeled open chromatin region DNA from the second labeled exposed closed chromatin region DNA and the second labeled open chromatin region DNA following the step of performing a DNA sequencing reaction to obtain the DNA sequence of the first labeled open chromatin region DNA, the second labeled exposed closed chromatin region DNA, or the second labeled open chromatin region DNA comprised within the chromosomal DNA. In yet another embodiment, the DNA sequence variation or epigenetic modification is associated with a condition selected from the group consisting of cancer, a genetic disease or condition, a developmental disease or condition, or an immunological disease or condition. In certain embodiments, the methods of the present disclosure may be referred to as wellDA-seq.

In certain embodiments, the methods of the present disclosure may comprise identifying the DNA sequence variation in the amplified second labeled exposed closed chromatin region DNA. The DNA sequence variation, in one embodiment, is selected from the group consisting of a mutation, a copy number alteration, a structural variation, and a DNA modification. Identifying the epigenetic modification comprises, in particular embodiments, performing a Cleavage Under Targets and Tagmentation (Cut&Tag) analysis of the open chromatin region DNA to measure protein:DNA interactions. In some embodiments, the methods of the present disclosure may comprise detecting at least one epigenetic modification in close proximity to the open chromatin region DNA. Identifying the epigenetic modification, in certain embodiments, comprises performing an Assay for Transposase-Accessible Chromatin (ATAC) analysis on the open chromatin region DNA to measure chromatin accessibility differences in the chromosomal DNA.

In yet another aspect, the present disclosure provides a kit comprising: a) a first transposase; b) a first adaptor molecule for labeling open chromatin region DNA; c) a second adaptor molecule for labeling closed chromatin region DNA and open chromatin region DNA; and d) a chromatin disruption agent for disrupting the chromatin structure of chromosomal DNA. In one embodiment, the kit further comprises a second transposase. In another embodiment, the kit further comprises a first set of primers for amplifying labeled open chromatin region DNA and a second set of primers for amplifying open chromatin region DNA and closed chromatin region DNA. In yet another embodiment, the first adaptor molecule or the second adaptor molecule further comprises a third label comprising an oligonucleotide barcode sequence. In still yet another embodiment, the first set of primers or the second set of primers further comprises a third label comprising an oligonucleotide barcode sequence.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 provides an example of a workflow of a method performed on a sample comprising low-input material, such as a low number of cells or nuclei or a single cell or nucleus, or a suspension thereof. The open chromatin region DNA of the sample is first labeled by tagmentation or by other methods known in the art to introduce a first set of oligonucleotides or barcodes. In specific embodiments, the tagmentation reaction may be performed using a Tn5 transposome, a mutated Tn5 transposome, an antibody attached Tn5 transposome, an engineered Tn5 transposome, or any other transposome known in the art. Then, the sample is lysed to fully or partially remove the chromatin. Following lysis, another set of oligonucleotides or barcodes, which is distinct from that used to label the open chromatin region DNA, is used to label closed chromatin region DNA as well as open chromatin region DNA by tagmentation or by other methods known in the art. Closed chromatin region DNA and open chromatin region DNA with their specific barcodes are then amplified through the subsequent PCR reaction. The primers used for amplifying closed chromatin region DNA and open chromatin region DNA can include oligonucleotide or barcode sequences, which are used as the sample barcode. Barcoded closed chromatin region DNA and open chromatin region DNA from a number of samples may then be pooled together following amplification. Closed chromatin region DNA and open chromatin region DNA libraries can be separated according to their specific barcodes, biotin modifications, or by other methods known in the art to prepare high-throughput sequencing libraries for downstream sequencing.

FIG. 2 provides an example of a workflow of a method performed on a sample comprising permeabilized, fixed, antibody stained, engineered, or labeled nuclei or cells. The open chromatin region DNA of the bulk sample is first labeled by tagmentation or by other methods known in the art to introduce a first set of oligonucleotides or barcodes. In specific embodiments, the tagmentation reaction may be performed using a Tn5 transposome, a mutated Tn5 transposome, an antibody attached Tn5 transposome, an engineered Tn5 transposome, or any other transposome known in the art. The bulk sample with labeled open chromatin region DNA is then sorted or dispensed into tubes, plates, or wells, such that one cell or nucleus is present per tube or well. Then, the single cell or nucleus in each tube or well is lysed to fully or partially remove the chromatin. Following lysis, another set of oligonucleotides or barcodes, which is distinct from that used to label the open chromatin region DNA, is used to label closed chromatin region DNA as well as open chromatin region DNA by tagmentation or by other methods known in the art. Closed chromatin region DNA and open chromatin region DNA with their specific barcodes are then amplified through the subsequent PCR reaction. The primers used for amplifying closed chromatin region DNA and open chromatin region DNA can include oligonucleotide or barcode sequences, which are used as the well, tube, or cell barcode. Barcoded closed chromatin region DNA and open chromatin region DNA from all reaction tubes or wells are then pooled together following amplification. Closed chromatin region DNA and open chromatin region DNA libraries can be separated according to their specific barcodes, biotin modifications, or by other methods known in the art to prepare high-throughput sequencing libraries for downstream sequencing.

FIG. 3 provides an example of a workflow of a method performed on a sample comprising permeabilized, fixed, antibody stained, engineered, or labeled nuclei or cells. The sample is first incubated with antibodies that bind to a targeted chromatin protein, then a second antibody is added that enhances the tethering of pA-Tn5 transposome to the antibody-bound sites. Next pA-Tn5 is activated with Mg⁺⁺ to perform the first tagmentation reaction to label open chromatin region DNA. After washing, the tagmented sample is then sorted or dispensed into tubes, plates, nanowells, chips, or microdroplets such that each tube, well, or droplet contains a single cell or nucleus. Then, the single cell or nucleus in each tube, well, or droplet is lysed to fully or partially remove chromatin structures. After that, another tagmentation reaction is performed to label the closed chromatin region DNA as well as open chromatin region DNA by a transposase carrying another set of oligonucleotides or barcodes, different from the oligonucleotides or barcodes used to label the open chromatin region DNA. The closed chromatin region DNA and the open chromatin region DNA with their specific barcodes are then amplified through the subsequent PCR reaction. The primers used for amplifying the closed chromatin region DNA and open chromatin region DNA can include barcode sequences, which are used as the well, tube, droplet, or cell barcode. The barcoded closed chromatin region DNA and open chromatin region DNA molecules from all wells, tubes, or droplets may then be pooled together following amplification. The closed chromatin region DNA and open chromatin region DNA libraries can be separated according to their specific barcodes, biotin modifications, or by other methods known in the art to prepare high-throughput sequencing libraries for downstream sequencing.

FIG. 4 provides an example of two different sets of Tn5 transposomes. In the first set (FIG. 4, Panel A), the Tn5 transposase combines two oligonucleotides (A and B) with mosaic end (ME) sequences. The nucleotide sequences of A and B can be distinct, or they can be the same. In specific embodiments, the two oligonucleotides can have barcode sequences and/or unique molecular identifier (UMI) sequences, such as those shown in panels 2 and 3 of FIG. 4, Panel A. In the second set (FIG. 4, Panel B), the Tn5 transposase combines another set of two oligonucleotides (C and D) with mosaic end (ME) sequences. The nucleotide sequences of C and D can be distinct, or they can be the same. These two oligonucleotides can have barcode sequences and/or unique molecular identifier (UMI) sequences, such as those shown in panels 2 and 3 of FIG. 4, Panel B. If these sets of Tn5 transposomes are used to label the open chromatin region DNA and the closed chromatin region DNA, respectively, then at least one of A or B is different from at least one of C or D.

FIG. 5 shows the results of a high-throughput nanowell-based method performed on cells of the MDA-MB-231 cancer cell line. FIG. 5, Panel A shows a Venn diagram demonstrating the number of cells for which information was obtained for open chromatin region DNA, closed chromatin region DNA, or both open chromatin region DNA and closed chromatin region DNA. FIG. 5, Panel B shows the ATAC clustering results using the chromatin accessibility profiles. FIG. 5, Panel C shows a uniform manifold approximation and projection (UMAP) of single cells according to their chromatin accessibility profiles. FIG. 5, Panel C shows a UMAP of single cells according to their DNA copy number aberration profiles, from left to right UMAP was color-coded by copy number subclones, copy number subclones, and ATAC clustering profiles. FIG. 5, Panel D shows a Heatmap of DNA copy number aberrations in single cells according to the closed chromatin region DNA and open chromatin region DNA data, with super clones (Super) and subclones (Sub) annotated based on the heatmap clustering results. The ATAC annotation bar summarizes the ATAC clustering data as shown in FIG. 5, Panel B.

FIG. 6 shows the results of a high-throughput nanowell-based method performed on cells from normal breast tissue. FIG. 6, Panel A shows chromatin accessibility quality control plots for the human breast tissue samples. Each dot represents one nanowell with fragments, and nanowells with over 1000 fragments and a transcription start site (TSS) enrichment score larger than 2 are considered to be cells. FIG. 6, Panel B shows a Venn diagram demonstrating the number of cells for which information was obtained for open chromatin region DNA, closed chromatin region DNA, or both open chromatin region DNA and closed chromatin region DNA. FIG. 6, Panel C shows a UMAP of 8 clusters of single cells, each of which represents a different cell type found in the normal human breast tissue identified by chromatin accessibility data. FIG. 6, Panel D shows the top 30 differentially expressed genes, inferred from the chromatin accessibility data, of each cluster and highlights three canonical markers. FIG. 6, Panel E shows a Heatmap of DNA copy number aberrations in single cells according to the closed chromatin region DNA and open chromatin region DNA data. The Heatmap shows one cluster of aneuploid cells and one cluster of diploid cells, and the ATAC side-bar summarizes the chromatin accessibility clustering data as shown in FIG. 6, Panel C. FIG. 6, Panel F shows the mapping of cells from DNA subclone s1 to the UMAP space of the open chromatin region DNA profiles and demonstrates that all 12 single cells from s1 were mapped to lumSec clusters.

FIG. 7 provides an example of a workflow that may be used according to certain embodiments of the present disclosure utilizing a nanowell chip. Single cell/nuclei suspensions are first tagmented with a first set of Tn5 (Tn5-A), then loaded into a nanowell chip with 5,184 nanowells. Then the chromatin is removed to expose closed DNA regions. Another set of Tn5 (Tn5-B) is used to perform tagmentation to label the closed chromatin regions, followed by PCR amplification with primers (72 rows×72 columns for ATAC; 72 rows×72 columns for DNA) that bind to both sets of Tn5 adaptors to assign cell barcodes to each molecule. Cell barcode assigned molecules of the whole chip are then pooled together. Further, the ATAC and DNA libraries are then enriched with library specific primers to construct high throughput sequencing libraries.

FIG. 8 shows the results of a DNA-Cut&Tag co-assay experiment. FIG. 8, Panel A shows DNA-Cut&Tag co-assay quality control plots for K562 cells, in which each dot represents a nanowell. FIG. 8, Panel B shows the fraction of reads in peaks, TSS, promoter, enhancer, and mitochodrial genes. FIG. 8, Panel C shows the number of cells in which Cut&Tag and/or DNA modalities were successfully profiled. FIG. 8, Panel D shows a track plot comparison with Cut&Tag data from the co-assay (inhouse) and a Cut&Tag assay from published data. FIG. 8, Panel E shows a heatmap of single cell DNA copy number profiles from the DNA-Cut&Tag co-assay.

FIG. 9 provides performance evaluation of a workflow that may be used according to certain embodiments of the present disclosure utilizing a nanowell chip. FIG. 9, Panel A shows quality control plots for a 10× (10× Genomics) experiment and two wellDA-seq experiments (wellDA-1 and wellDA-2). Each dot represents one well. FIG. 9, Panel B shows a comparison of the aggregated counts per million (CPM) fragments within peaks for the MDA-MB-231 cell line in the 10×, wellDA-1, and wellDA-2 experiments by Pearson correlation coefficient R and p value (p). FIG. 9, Panel C shows a comparison of single cell ATAC-seq (scATAC) profiles from the 10×, wellDA-1, and wellDA-2 experiments in a region of chromosome 2. The upper panels show the aggregated profiles of all cells, and the lower panels show fragments present in each of the 20 random cells. FIG. 9, Panel D shows a comparison of overdispersion metrics for the genomic bin counts and breadth of coverage metrics for wellDA-seq and four other scDNA-seq methods. Coverage was calculated from 120 randomly sampled cells per method and using 750K reads per cell as input. FIG. 9, Panel E shows the number of cells with DNA and/or ATAC data that were profiled by the two wellDA-seq experiments. FIG. 9, Panel F shows a UMAP showing the clustering results of single cell DNA copy number profiles from wellDA-seq, colored by DNA subclones or ATAC clusters (top panel), and a UMAP showing the clustering results of single cell ATAC-seq profiles from wellDA-seq, colored by ATAC clusters or DNA subclones (bottom panel). FIG. 9, Panel G shows single cell copy number heatmaps from two merged wellDA-seq experiments. FIG. 9, Panel H shows a UMAP showing the inferred single cell DNA copy number profiles from single cell ATAC-seq data of wellDA-seq. Pearson correlation of single cell DNA copy number profiles from the DNA modality of wellDA-seq and the inferred single cell DNA copy number profiles from the ATAC modality are shown in FIG. 9, Panel I.

FIG. 10 shows the results of simultaneously profiling of the single cell copy number and single cell ATAC-seq from two normal human breast tissues using wellDA-seq. FIG. 10, Panel A shows a UMAP of scATAC-seq data of wellDA-seq from the first normal breast tissue, which identified 8 different cell types. FIG. 10, Panel B shows a heatmap of single cell DNA copy number profiles of wellDA-seq, in which 1418 single cells were profiled. The bottom panel shows the integer copy number of two different subclones identified by the singe cell DNA data. FIG. 10, Panel C shows that the aneuploid cells from subclone c1 map to the UMAP of scATAC-seq, which identified luminal secretory (LumSec) as the cell population harboring the somatic copy number alteration (CNA) events. FIG. 10, Panel D shows a UMAP of scATAC-seq data of wellDA-seq for the 2nd normal breast tissue. FIG. 10, Panel E shows a heatmap of single cell DNA copy number profiles of wellDA-seq, in which 962 single cells were profiled. FIG. 10, Panel F shows the aneuploid cells from subclone c1-c3 map to the UMAP of scATAC-seq, which identified LumSec, luminal hormone responsive (LumHR), and fibroblast as the cell populations that harbor different somatic CNA events.

FIG. 11 shows an overview of single cell ATAC data and single cell DNA (scDNA) data of wellDA-seq profiled from 9 breast cancer patients. FIG. 11, Panel A shows an integrated UMAP of scATAC profiles from 9 breast cancer patients, colored by cell types. In total, 11 cell types from 19,334 cells were identified. FIG. 11, Panel B shows an integrated UMAP of scATAC profiles from 9 breast cancer patients, colored by patient. FIG. 11, Panel C shows the cell number and proportion of each cell type in each patient. FIG. 11, Panel D shows the consensus integer copy number profiles of 73 subclones identified by wellDA-seq from the 9 patients.

FIG. 12 shows gene dosage effects of subclonal copy number alteration (CNA) events on chromatin accessibility. FIG. 12, Panel A shows a heatmap showing the single-cell CNA profiles of the representative sample 66T. Color bars denote the clones. FIG. 12, Panel B shows a UMAP showing the single cells based on the ATAC (left panel) and CNA (right panel) profiles. Dots (cells) are colored by the clones labeled by the cell counts. FIG. 12, Panel C shows a minimum evolution tree constructed by MEDIC2. Size of nodes denotes the frequency of cells of clones. FIG. 12, Panel D shows a heatmap showing the consensus integer CNAs of two presentative clones, C1 and C4, along genomic Varbins (columns). The bar annotations show if a genomic Varbin has a CNA event (CNA), a peak with open chromatin (ATAC), a differentially aneuploid bin (DAB), a differentially accessible ATAC peak (DAP), and a differentially accessible chromatin hub (DACH). FIG. 12, Panel E shows a Venn diagram (top panel) showing the DABs and DACHs in a comparison of the clone C1 and C4. The bottom panel shows a diagram showing the scheme of calculating genome to epigenome (GtoE) and epigenome by genome (EbyG) percentages. FIG. 12, Panel F shows a UMAP of the single-cell ATAC profiles that are colored by the clones (left panel) and the module scores of the DACHs in a comparison of C1 and C4. FIG. 12, Panel G shows a lollipop plot showing the significant gene signatures that were enriched for the DACHs in a comparison of C1 and C4. FIG. 12, Panel H shows a track plot at a 200 kb window of the TSS of the gene IGF1R showing the copy number (top panel), the normalized ATAC fragments (second panel), and the presence of Tn5 insertions in randomly selected cells (third panel), and the genes and peaks (bottom panel). FIG. 12, Panel I shows a boxplot showing the GtoE percentage of the samples. FIG. 12, Panel J shows a boxplot showing the EbyG percentage in the samples. FIG. 12, Panel K shows a scatter plot comparing the count of Varbins with DACHs and DABs. A Pearson correlation coefficient was calculated, and the p-value is labeled. In FIG. 12, Panels I, J, K, each dot denotes a clone-to-clone comparison.

### BRIEF DESCRIPTION OF THE SEQUENCES

SEQ ID NO:1—A representative mosaic end nucleotide sequence that is recognized by a transposase.

SEQ ID NO:2—A representative nucleotide adaptor sequence that may be used in the assembly of a Tn5 transposome.

SEQ ID NO:3—A representative nucleotide sequence that may be used in the assembly of a Tn5 transposome.

SEQ ID NO:4—A representative nucleotide adaptor sequence that may be used in the assembly of a Tn5 transposome.

SEQ ID NO:5—A representative nucleotide adaptor sequence that may be used in the assembly of a Tn5 transposome.

SEQ ID NO:6—A representative nucleotide adaptor sequence that may be used in the assembly of a Tn5 transposome.

SEQ ID NO:7—The nucleotide sequence of a representative PCR primer that may be used in an amplification reaction to amplify tagmented DNA.

SEQ ID NO:8—The nucleotide sequence of a representative PCR primer that may be used in an amplification reaction to amplify tagmented DNA.

SEQ ID NO:9—The nucleotide sequence of a representative PCR primer that may be used in an amplification reaction to amplify tagmented DNA.

SEQ ID NO:10—The nucleotide sequence of a representative PCR primer that may be used in an amplification reaction to amplify tagmented DNA.

SEQ ID NO:11—The nucleotide sequence of a representative PCR primer that may be used in an amplification reaction to amplify tagmented DNA.

SEQ ID NO:12—The nucleotide sequence of a representative PCR primer that may be used in an amplification reaction to amplify tagmented DNA.

SEQ ID NO:13—The nucleotide sequence of a representative PCR primer that may be used in an amplification reaction to amplify tagmented DNA.

### DETAILED DESCRIPTION OF THE INVENTION

The present disclosure provides a novel method that for the first time allows for approximately simultaneous profiling of closed chromatin region DNA and open chromatin region DNA from single cells or from low-input material. In certain embodiments, the methods provided by the present disclosure may be referred to as wellDA-seq. The methods provided herein can be adapted to low-, mid- and high-throughput applications for closed chromatin region DNA and open chromatin region DNA co-amplification and DNA library preparation. The methods of the present disclosure, in particular embodiments, utilize two different sets of adaptors to separately label the open chromatin region DNA and the closed chromatin region DNA from single cells or from low-input material. In one embodiment, the method utilizes different sets of oligonucleotides or barcodes to label the closed chromatin region DNA and the open chromatin region DNA and then pools all of the barcoded amplified products together to prepare the closed chromatin region DNA and the open chromatin region DNA libraries approximately simultaneously. The methods provided by the present disclosure thus overcome many of the challenges associated with the bulk sequencing methods currently known in the art, which are limited to profiling a large population of cells, and thus only provide an average measurement over the entire population. This masks the variation present in complex tissues. These limitations impair the ability to determine how genetic mutations and variations impact the epigenetic landscape.

### A. Method Overview

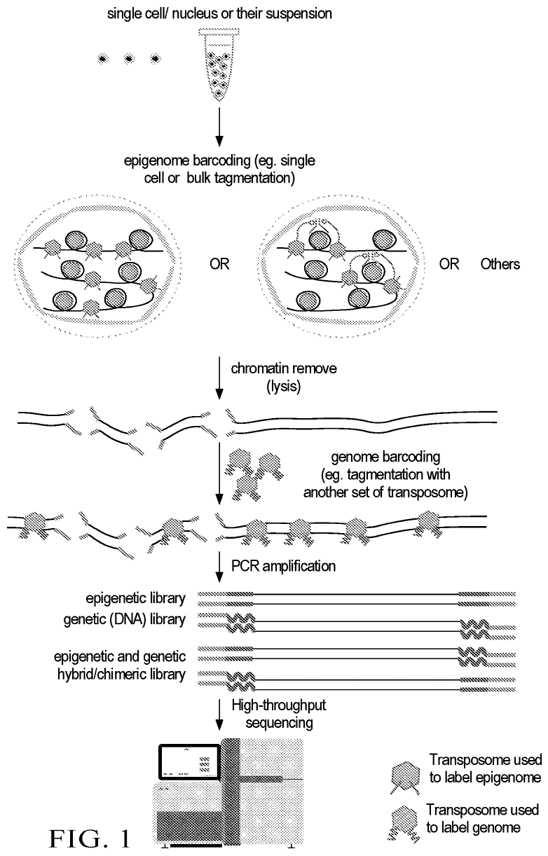

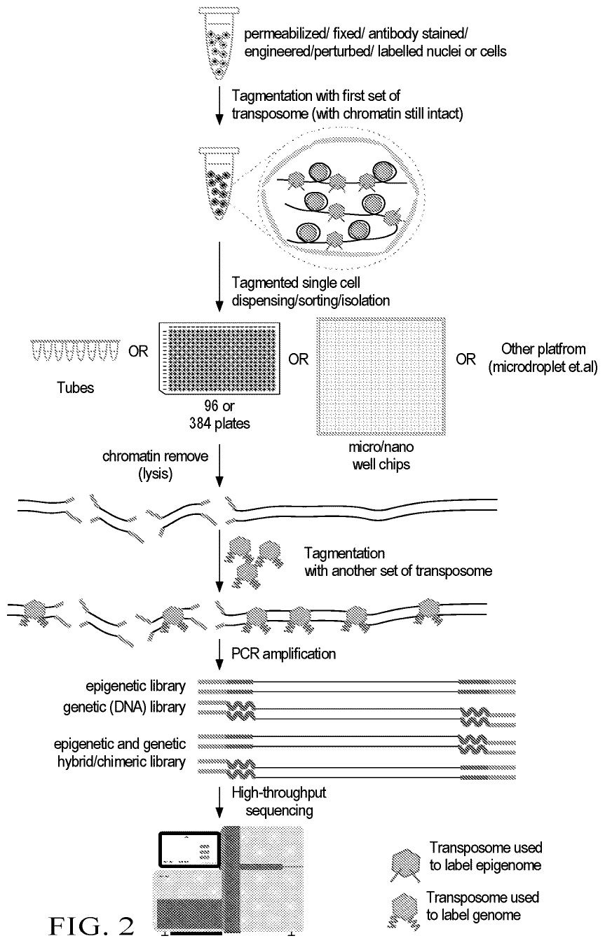

The present disclosure provides methods that allow for the independent labeling of closed chromatin region DNA and open chromatin region DNA followed by amplification of the labeled or barcoded materials approximately simultaneously, without physical separation of the closed chromatin region DNA and the open chromatin region DNA prior to amplification (FIGS. 1-3). The methods provided by the present disclosure may be used to approximately simultaneously amplify open chromatin region DNA and closed chromatin region DNA from a single cell obtained from a sample comprising about 1 cell to about 1,000,000,000 cells, including all ranges derivable therebetween. The sample may comprise, for example, about 1 cell to about 1,000,000,000 cells, about 1 cell to about 500,000,000 cells, about 1 cell to about 100,000,000 cells, about 1 cell to about 50,000,000 cells, about 1 cell to about 10,000,000 cells, about 1 cell to about 5,000,000 cells, about 1 cell to about 1,000,000 cells, about 1 cell to about 500,000 cells, about 1 cell to about 100,000 cells, about 1 cell to about 50,000 cells, about 1 cell to about 10,000 cells, about 1 cell to about 5,000 cells, about 1 cell to about 1,000 cells, about 1 cell to about 500 cells, about 1 cell to about 100 cells, about 1 cell to about 50 cells, about 1 cell to about 25 cells, or about 1 cell to about 10 cells, including all ranges derivable therebetween. In some embodiments, labeled open chromatin region DNA and labeled closed chromatin region DNA from all samples or sub-samples may be pooled together prior to amplification. In certain embodiments, the labeled open chromatin region DNA and labeled closed chromatin region DNA libraries may be separated after pooling all of the libraries of all samples or sub-samples to construct the closed chromatin region DNA and the open chromatin region DNA libraries individually. The closed chromatin region DNA and the open chromatin region DNA libraries may then, in some embodiments, be separated prior to high-throughput sequencing. In certain embodiments, methods of the present disclosure may include one or more of the steps of: (1) labeling the open chromatin region DNA with one set of DNA barcodes or labels when the chromatin is still intact; (2) partially or fully removing chromatin from the DNA; (3) labeling the closed chromatin region DNA and the open chromatin region DNA using another set of DNA barcodes or labels; (4) amplifying the closed chromatin region DNA and the open chromatin region DNA through the aforementioned closed chromatin region DNA and open chromatin region DNA barcodes or labels to add cell- or sample-specific barcodes or labels to the closed chromatin region DNA and the open chromatin region DNA in each tube, well, or nanodroplet; (5) combining all of the labeled closed chromatin region DNA and labeled open chromatin region DNA libraries from each tube, well, or droplet; (6) separating the closed chromatin region DNA and the open chromatin region DNA libraries based on molecular features; (7) preparing the closed chromatin region DNA and the open chromatin region DNA libraries for sequencing, if needed; and (8) sequencing the closed chromatin region DNA and the open chromatin region DNA libraries and analyzing the sequencing data. In some embodiments, since different barcodes, adaptors, or labels are used to label the closed chromatin region DNA and the open chromatin region DNA, the methods provided herein can be used to enrich one of these modalities, for example the open chromatin region DNA, as desired prior to or after exponential co-amplification. This step may be performed, in certain embodiments, when the concentration of one modality concentration is significantly less than the other. In particular embodiments, the analysis of sequencing data may include computationally matching the data from the closed chromatin region DNA and open chromatin region DNA and performing a more detailed analysis.

The methods of the present disclosure are able to add a unique sample or cell barcode to closed chromatin region DNA and open chromatin region DNA using a multiplexing PCR reaction. This allows for the identification of closed chromatin region DNA and open chromatin region DNA of each cell or sample following amplification. The methods provided herein thus allow for the combined preparation of closed chromatin region DNA and open chromatin region DNA sequencing libraries from all of the samples, sub-samples, or cells together. This significantly reduces the labor and resource input required to prepare individual closed chromatin region DNA and open chromatin region DNA libraries from each cell, sample, or sub-sample one at time. This feature of the methods of the present disclosure makes them highly scalable to tube format, plate format, or high-throughput platforms such as nanowells, nanochips, or nanodroplets.

The methods of the present disclosure have broad application in cancer genomics, single cell genomics, pre-natal genetic diagnosis, drug-target discovery, forensics, neurological disease, neuroscience, microbiology, pathogenesis, and development. The methods provided by the present disclosure additionally have broad application in low-input closed chromatin region DNA and open chromatin region DNA library construction and highly multiplexed open chromatin region DNA and closed chromatin region DNA library preparation from single cells, multiple cells, or cell mixtures. The methods provided by the present disclosure can also be used in many clinical and translational applications such as early disease diagnosis, disease monitoring, minimal residual disease detection, novel predictive and prognostic biomarkers development, and novel actionable target identification using samples that may include, but are not limited to, small chunks or pieces of tissue, blood droplets, buffy coat, body fluids, swabs, and patient-derived materials such as organoids and patient-derived xenografts.

### B. Nucleosomes and the Epigenome

Nucleosomes are the primary scaffold of chromatin folding, and as such have a significant influence on chromatin structure. Nucleosome positioning in the genome has a regulatory function and significantly affects the in vivo availability of transcription factor binding sites to transcription factors and the general transcription machinery. As such, nucleosome positioning regulates DNA-dependent processes such as transcription, DNA repair, replication, and recombination. Nucleosome positioning also has an effect on genomic instability in healthy and diseased tissues, such as cancer. The nucleosome core consists of the histone proteins, H2A, HB, H3 and H4, and histone acetylation influences nucleosome structure and arrangement. Histone acetylation relaxes the chromatin structure, resulting in activation of gene transcription. In contrast, deacetylated chromatin is generally transcriptionally inactive. Furthermore, methylation of DNA at CpG dinucleotides is enriched in closed chromatin regions and depleted in open chromatin regions. In the human genome, an increase in methylated CpG density correlates with nucleosome occupancy (Collings, et al., Epigenetics & Chromatin 10:18, 2017). As used herein the term “chromatin” refers to the complex of DNA and proteins that form the chromosomes of eukaryotic cells. As used herein the term “open chromatin region,” “open chromatin,” or “open chromatin region DNA” refers to a nucleosome depleted region of DNA that can be accessed by DNA regulatory elements. DNA regulatory elements have limited or no access to regions of nucleosome dense regions of DNA. These regions are referred to herein as a “closed chromatin region,” “closed chromatin,” or “closed chromatin region DNA.” An amplified open chromatin region DNA product may be, in certain embodiments, from a chromatin region comprising acetylated histones or decreased DNA methylation, whereas an amplified closed chromatin region DNA product may be, in particular embodiments, be from a chromatin region comprising deacetylated histones or increased DNA methylation. In some embodiments, an open chromatin region may refer to a chromatin region that is transcriptionally active, whereas a closed chromatin region may refer to a chromatin region that is transcriptionally inactive. Amplified open chromatin region DNA and closed chromatin region DNA products can be used to directly measure the effect of chromatin structure, and modifications thereof, on gene transcription and to study the regulatory function of nucleosome positioning. These amplified products may additionally be used to study DNA binding and regulation of the general transcription machinery, and how certain genomic regions regulate gene expression. The amplified open chromatin region DNA and closed chromatin region DNA products may additionally be used to identify cell types and cell states, to investigate the role of tumor-associated immune cells, to detect disease-associated changes in chromatin structure and transcription regulation, to analyze the population specific chromatin accessibility, to investigate the interactions between proteins and DNA, to investigate DNA binding sites of a protein of interest, and to examine gene regulation and chromatin-associated protein binding.

### C. Samples and Sample Preparation

The methods of the present disclosure are able to amplify closed chromatin region DNA and open chromatin region DNA approximately simultaneously from material derived from single cells, tens of cells, hundreds of cells, thousands of cells, or millions of cells. For example, closed chromatin region DNA and open chromatin region DNA may be approximately simultaneously amplified from material derived from about 1 cell to about 1,000,000,000 cells, about 1 cell to about 500,000,000 cells, about 1 cell to about 100,000,000 cells, about 1 cell to about 50,000,000 cells, about 1 cell to about 10,000,000 cells, about 1 cell to about 5,000,000 cells, about 1 cell to about 1,000,000 cells, about 1 cell to about 500,000 cells, about 1 cell to about 100,000 cells, about 1 cell to about 50,000 cells, about 1 cell to about 10,000 cells, about 1 cell to about 5,000 cells, about 1 cell to about 1,000 cells, about 1 cell to about 500 cells, about 1 cell to about 100 cells, about 1 cell to about 50 cells, about 1 cell to about 25 cells, or about 1 cell to about 10 cells, including all ranges derivable therebetween. In some embodiments, closed chromatin region DNA and open chromatin region DNA may be approximately simultaneously amplified from extracted, unextracted, purified, or isolated chromatin. The cell or cells from which open chromatin region DNA and closed chromatin region DNA are approximately simultaneously amplified may be from any eukaryotic organism. Non-limiting examples of such organisms include eukaryotic single-celled organisms, eukaryotic multicellular organisms, fungi, plants, animals, reptiles, amphibians, insects, mammals, and humans. Samples for use in the methods provided by the present disclosure may be derived from any eukaryotic cell or tissue, non-limiting examples which include chunks or pieces of tissue, blood droplets, buffy coats, body fluids, tissue swabs, organoids, and patient-derived xenografts.

In some embodiments, a sample comprising open chromatin region DNA and closed chromatin region DNA may comprise a plurality of cells. For example, the sample may comprise at least 2, at least 5, at least 10, at least 25, at least 50, at least 100, at least 500, at least 1,000, at least 5,000, at least 10,000, at least 20,000, at least 30,000, at least 40,000, at least 50,000, at least 60,000, at least 70,000, at least 80,000, at least 90,000, at least 100,000, at least 200,000, at least 300,000, at least 400,000, at least 500,000, at least 600,000, at least 700,000, at least 800,000, at least 900,000, at least 1,000,000, or at least 10,000,000 cells, including all ranges derivable therebetween. In particular embodiments, the sample may be separated into a plurality of sub-samples and each sub-sample may comprise about 1 cell to about 50 cells, about 1 cell to about 40 cells, about 1 cell to about 30 cells, about 1 cell to about 20 cells, about 1 cell to about 15 cells, about 1 cell to about 10 cells, or about 1 cell to about 5 cells, including all ranges derivable therebetween. In specific embodiments, each sub-sample may comprise a single cell. Methods of separating cell suspensions are known in the art and any such method may be used according to the methods provided by the present disclosure. Non-limiting examples of such methods include flow cytometry and cell distribution using single-cell microdispensers.

Cells or nuclei for use in the present disclosure may be permeabilized, fixed, antibody-stained, engineered, perturbed, modified, or labeled. Methods for cell permeabilization, fixation, antibody-staining, engineering, modifying, and labeling are known in the art and any such method may be used according to the methods provided by the present disclosure. For example, cells or nuclei may be permeabilized using organic solvents, non-limiting examples of which include acetone and methanol, or by using detergents, non-limiting examples of which include saponin, Triton X-100, and Tween-20. Cell or nuclei fixation may be performed using chemical or physical methods. For example, cells may be fixed using cross-linking agents such as formaldehyde, glutaraldehyde, and succinimide esters, or by using solvents. In certain embodiments, cells may be fixed using heat, microwaving, or cryopreservation methods. Cells or nuclei for use in the present disclosure may, in some embodiments, be immunostained, engineered to express a protein of interest, or labeled using, for example, a fluorescent dye. In particular embodiments, cell or nuclei for use in the present disclosure may be prepared into a single cell or single nuclei suspension.

### D. Open Chromatin Region DNA Labeling

To approximately simultaneously prepare closed chromatin region DNA and open chromatin region DNA libraries, the open chromatin region DNA, or a portion thereof, must first be labeled with a first set of labels. These labels may be, for example, a set of oligonucleotides or barcodes. As used herein, the term “label” refers to a directly or indirectly detectable oligonucleotide or nucleotide modification that is conjugated directly or indirectly to the composition to be detected. Non-limiting examples of a nucleotide modification that may be present, in certain embodiments, in a label as described herein include DNA methylation, a biotin label, a fluorescent label, or a chemical modification. Methods to introduce oligonucleotide labels or barcodes are known in the art and any such method may be used according to the methods provided by the present disclosure. As used herein the terms “oligonucleotide:” “polynucleotide,” and “nucleic acid” may be used interchangeably and include linear oligomers of natural or modified monomers or linkages. An oligonucleotide may include, for example, deoxyribonucleosides, ribonucleosides, α-anomeric forms thereof, peptide nucleic acids, and the like, capable of specifically binding to a target polynucleotide by way of a regular pattern of monomer-to-monomer interactions, such as Watson-Crick type of base pairing, base stacking, Hoogsteen, or reverse Hoogsteen type base pairing. Monomers may be linked, in some embodiments, by a phosphodiester bond or an analog thereof to form oligonucleotides ranging in size from a few monomeric units to several tens of monomeric units. Whenever an oligonucleotide is represented by a sequence of letters herein, a person of ordinary skill in the art would understand that the nucleotides are in 5′ to 3′ orientation from left to right. A person of ordinary skill in the art would further understand that if an oligonucleotide is presented as a sequence of letters that “A” denotes adenine, “C” denotes cytosine, “G” denotes guanine, and “T” denotes thymine, and “U” denotes uracil, unless otherwise noted. Analogs of phosphodiester linkages include, but are not limited to, phosphorothioate, phosphorodithioate, phosphoranilidate, and phosphoramidate linkages. It is clear to those skilled in the art when oligonucleotides having natural or non-natural nucleotides may be employed. For example, a person of ordinary skill in the art would understand when processing by enzymes may be employed or when oligonucleotides consisting of natural nucleotides are required.

A nucleic acid for use in the methods of the present disclosure may be a modified or unmodified and may comprise RNA nucleotides or DNA nucleotides. In some embodiments, a nucleic acid molecule for use in the present disclosure is an unmodified oligonucleotide. An unmodified oligonucleotide may be composed, in certain embodiments, of nucleobases, sugars, and covalent internucleoside linkages. The term “oligonucleotide analog” as used herein refers to oligonucleotides that have one or more non-naturally occurring segments. As used herein the term oligonucleotide also includes oligonucleotide analogs. Oligonucleotide analogs may function, in certain embodiments, similarly, to naturally occurring oligonucleotides. A person of ordinary skill in the art would understand that oligonucleotide analogs may, in some embodiments, have desirable properties such as, for example, enhanced cellular uptake, enhanced affinity for other oligonucleotide or nucleic acid targets, or increased stability in the presence of nucleases. In certain embodiments, oligonucleotides for use in the present disclosure may comprise modified or non-naturally occurring internucleoside linkages. Such non-naturally internucleoside linkages may, in particular embodiments, confer desired properties to the oligonucleotide, non-limiting examples of which include, enhanced cellular uptake, enhanced affinity for other oligonucleotide or nucleic acid targets, or increased stability in the presence of nucleases. Types of non-naturally occurring internucleoside linkages are known in the art and any such linkage may be used according to the methods of the present disclosure. Non-limiting examples of such non-naturally occurring or modified internucleoside linkages include internucleoside linkages that retain a phosphorus atom and internucleoside linkages that do not have a phosphorus atom. In certain embodiments, non-naturally occurring or modified internucleoside linkages that do not include a phosphorus atom may comprise internucleoside linkages that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having amide backbones; and others, including those having mixed N, O, S and CH₂ components.

In particular embodiments, oligonucleotides may also include oligonucleotide mimetics. An oligonucleotide mimetic may include, for example, an oligonucleotide wherein only the furanose ring or both the furanose ring and the internucleotide linkage are replaced with a novel group. In one embodiment, the furanose ring may be replaced, for example, with a morpholino ring or any other sugar surrogate known in the art. In certain embodiments, an oligonucleotide mimetic may comprise one or more peptide nucleic acids or cyclohexenyl nucleic acids (Wang et al., J. Am. Chem. Soc. 122:8595-8602, 2000). In another embodiment, an oligonucleotide mimetic may be a phosphonomonoester nucleic acid, which incorporates a phosphorus group in the backbone. In yet another embodiment, an oligonucleotide mimetic may include replacement of the furanosyl ring with a cyclobutyl moiety.

In certain embodiments, oligonucleotides of the present disclosure may comprise one or more modified or substituted sugar moieties. Such modified or substituted sugars may, in some embodiments, improve stability in the presence of nucleases or binding affinity. Non-limiting examples of modified or substituted sugars include carbocyclic or acyclic sugars, sugars having substitute groups at one or more of their 2′, 3′ or 4′ positions, sugars having substitutes in place of one or more hydrogen atoms of the sugar, and sugars having a linkage between any two other atoms in the sugar. A large number of sugar modifications are known in the art and any such sugar modification may be used according to the present disclosure.

In some embodiments, oligonucleotides may include one or more nucleobase modifications or substitutions which are structurally distinguishable from, yet functionally interchangeable with, naturally occurring or synthetic unmodified nucleobases. Modified nucleobases may include in certain embodiments, synthetic or natural nucleobases such as 5-methylcytosine (5-me-C), 5-hydroxymethyl cytosine, 7-deaza-guanine, or 7-deaza-adenine, 2-aminopyridine, 2-pyridone, 5-substituted pyrimidines, 6-azapyrimidines and N-2 substituted purines, N-6 substituted purines, 0-6 substituted purines, 2 aminopropyladenine, 5-propynyluracil, and 5-propynylcytosine.

An oligonucleotide of the present disclosure may be, in certain embodiments, about 10 to about 1000, about 10 to about 900, about 10 to about 800, about 10 to about 700, about 10 to about 600, about 10 to about 500, about 10 to about 400, about 10 to about 350, about 10 to about 300, about 10 to about 250, about 10 to about 200, about 10 to about 150, about 10 to about 100, about 10 to about 50, about 10 to about 40, about 10 to about 30, or about 10 to about 20 nucleotides in length, including all ranges derivable therebetween.

The oligonucleotides of the disclosure, in certain embodiments, may comprise a barcode region, which can be used to identify a cellular characteristic. The barcode region can be a polynucleotide of at least, at most, about, or exactly 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, or 200 nucleotides in length. The barcode may comprise, in some embodiments, one or more universal PCR regions, adaptors, such as adaptors for making cDNA libraries, linkers, or a combination thereof. The barcode region may also include, in particular embodiments, a molecular index region (MI) which can be used to count how many barcode sequences are delivered into each cell or nucleus. The MI may be 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200 or more nucleotides in length, including all ranges derivable therebetween.

Non-limiting cellular characteristics that can be identified by the barcode region include sample identity, sub-sample identity, cell identity, nucleus identity, and whether the samples comprise open chromatin region DNA, closed chromatin region DNA, or both open chromatin region DNA and closed chromatin region DNA. In some embodiments, the barcode may be specific for open chromatin region DNA, closed chromatin region DNA, open and closed chromatin region DNA, a cell, a nucleus, or a population of cells or nuclei, such that isolation of sequencing of the barcode after combining multiple differentially barcoded DNA molecules, cells, samples, sub-samples, or nuclei identifies the cellular characteristic of the DNA molecules, cells, samples, sub-samples, or nuclei. The cellular characteristic, in certain embodiments, can then be associated with other sequencing data or analysis. For example, the analysis may include epigenomic, genomic, or transcriptomic information obtained by single-cell analysis of mRNA or DNA. In one embodiment, the barcode is unique to one cell. In particular embodiments, the barcode is unique to a population of cells, such as about 2, 3, 4, 5, 6, 7, 8, 9, 10, 50, 100, 500, 1,000, 5,000, 10,000, 25,000, 50,000, 100,000, 500,000, or 1,000,000 cells, including all ranges derivable therebetween.

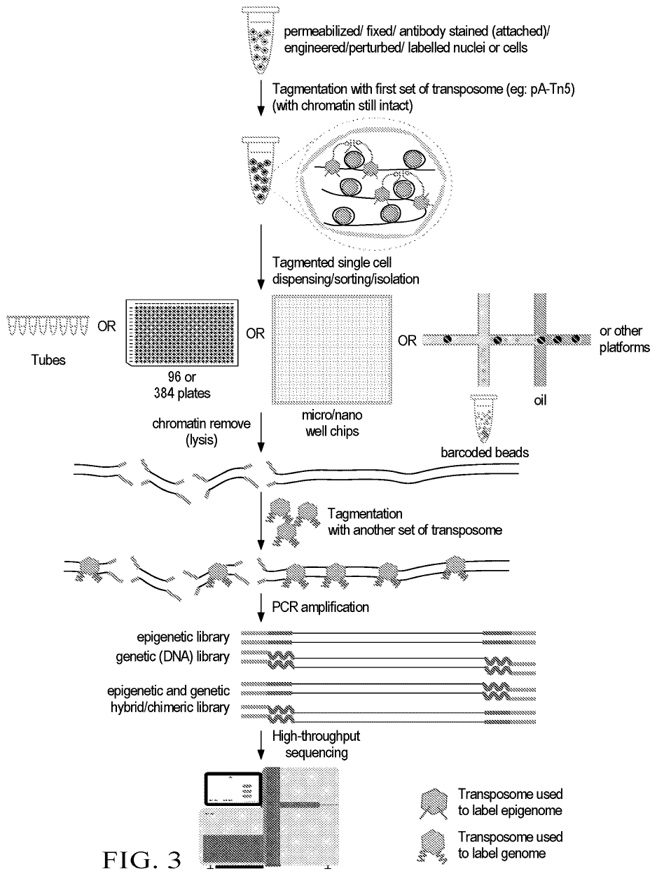

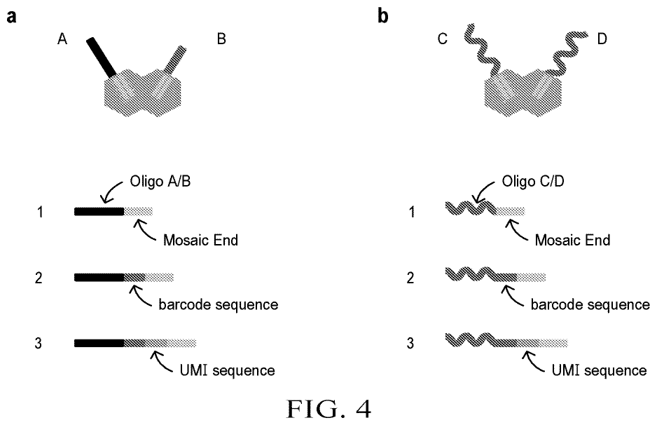

In particular embodiments, oligonucleotide labels or barcodes may be introduced through a tagmentation reaction. In summary, methods of tagmentation may include the use of enzymes known as transposases, which randomly cut DNA into short segments, known as “tags”. Adapter nucleotide sequences are the added to either side of the cut points through ligation. The adaptor oligonucleotide molecules may comprise nucleotide barcodes and/or primer binding sites for detection and amplification of the open chromatin region DNA sequences. Methods of tagmentation are known in the art and are further described in Zahn, H., et al., Nature Methods 14:167, 2017, which is incorporated herein by reference. Any transposome known in the art may be used according to the methods provided herein. Non-limiting examples of such transposomes include a Tn5 transposome, a mutated Tn5 transposome, an antibody attached Tn5 transposome, an engineered Tn5 transposome, a Mu transposome, or a sleeping beauty transposome. In one embodiment, the transposome is an engineered or modified Tn5, such as pA-Tn5 used in the Cut&Tag method described in Kaya-Okur, et al., Nature Communications 10:1930, 2019, which is specifically incorporated herein by reference (FIG. 3). In particular embodiments, the transposome may be loaded with universal oligonucleotides, or the transposome can be assembled by combining the transposase with transposase recognized DNA oligonucleotides. Transposase recognized oligonucleotides may include, in certain embodiments, oligonucleotides that comprise mosaic end (ME) sequences. Mosaic end sequences are known in the art and any such mosaic end sequence may be used according to the methods of the present disclosure. A non-limiting example of a mosaic end sequence includes the sequence of SEQ ID NO:1 (AGATGTGTATAAGAGACAG). In particular embodiments, the oligonucleotides that attach to the transposase can comprise a nucleotide barcode sequence, or a portion thereof, for distinguishing individual cells. In certain embodiments, the attached oligonucleotide sequence, or the portion thereof, can serve as the identifier for distinguishing the closed chromatin region DNA and open chromatin region DNA from the same single cell, nuclei, or sample (FIG. 4). In some embodiments, the oligonucleotide barcode sequence can comprise unique molecular identifier (UMI) sequences to distinguish individual molecular reads from PCR duplicate reads.

In certain embodiments, cells or nuclei comprising chromatin structure may be used to label the open chromatin region DNA, or a portion thereof, and to perform reactions, such as PCR and/or ligation, or to add the barcode oligonucleotides for distinguishing open chromatin region DNA from closed chromatin region DNA. The cell or nuclei suspension with barcode labeled open chromatin region DNA may then, in certain embodiments, be sorted by flow cytometry or dispensed by single cell microdispensers into a reaction chamber, tube, plate, well, nanowell, or nanochip, or embedded into microdroplets.

In certain embodiments, open chromatin region DNA may be labeled by ATAC, as described in Buenrostro, et al., Curr Protoc Mol Biol 109:21.29.1-21.29.9, 2015 or Buenrostro, et al., Nature Methods 10:1213-1218, 2013, which are specifically incorporated herein by reference. Briefly, cells may be harvested and pelleted by centrifugation, washed, and lysed with a non-ionic detergent to generate a nuclei preparation. Then the preparation may then incubated with a transposition mixture comprising a transposase and incubated at 37° C. and amplified in an amplification reaction using appropriate primers.

### E. Chromatin Disruption

Following labeling of the open chromatin region DNA, in some embodiments, the chromatin structure of the single sample, sub-sample, nucleus, or cell in each reaction chamber may be disrupted to remove or partially remove chromatin from the DNA. In particular embodiments, the cell, cells, nuclei, or nucleus present in reach reaction may be fully or partially lysed to remove or partially remove chromatin from the DNA. Methods for cell and nuclei lysis are known in the art and any such method may be used according to the methods of the present disclosure. As non-limiting examples, cell or nuclei lysis may be performed using an enzyme-based method, for example using a protease, a chemical-based method, for example using detergents such as Tween-20 or Triton X-100, a mechanical-based method, an acoustic-based method, an electrical-based method, or a combination of any of the aforementioned methods. Chromatin disruption methods provided by the present disclosure digest the cellular and/or nuclear membrane and disrupts chromatin structure to expose the DNA present within the structure.

### F. Closed Chromatin Region DNA Labeling

Following chromatin disruption, in some embodiments, the lysate may be used to perform a second labeling reaction to label the closed chromatin DNA with a second label. In particular embodiments, a tagmentation reaction may be performed using different oligonucleotide adaptors or nucleotide barcodes than those used for labeling the open chromatin region DNA prior to chromatin disruption. The transposome used to perform the second labeling reaction may be different from the one used to perform the first labeling reaction, or the transposome used to perform the second labeling reaction may be the same as the transposome used to perform the first labeling reaction. In certain embodiments, the transposome may be loaded with universal oligonucleotides, or the transposome may be assembled by combining the transposase with transposase recognized DNA oligonucleotides. In one embodiment, the transposase recognized oligonucleotides may comprise mosaic end (ME) sequences. The oligonucleotide sequences that attach to the transposase, in particular embodiments, may comprise a nucleotide barcode sequence, or a portion thereof, for distinguishing individual cells. In certain embodiments, the attached oligonucleotide sequence, or the portion thereof, can serve as the identifier for distinguishing the closed chromatin region DNA and open chromatin region DNA from the same single cell, nuclei, or sample. In particular embodiments, and as shown in FIGS. 1-3, the second labeling reaction may label open chromatin region DNA in addition to closed chromatin region DNA. Open chromatin region DNA may be distinguished from closed chromatin region DNA, however, because open chromatin region DNA will additionally comprise, in some embodiments, the first label introduced during the first labeling reaction, whereas closed chromatin region DNA will not additionally comprise this first label. In some embodiments, the oligonucleotide barcode sequence can comprise unique molecular identifier (UMI) sequences to distinguish individual molecular reads from PCR duplicate reads.

In some embodiments, the DNA fragments obtained after disruption of chromatin structure can also be used to perform reactions, such as PCR or ligation, to add the oligonucleotide or barcode sequences in order to distinguish closed chromatin region DNA from open chromatin region DNA. In certain embodiments, oligonucleotides used to perform ligation reactions may be phosphorylated, for example, at their 5′ end.

### G. Amplification Reactions

The methods provided by the present disclosure utilize different adaptor or oligonucleotide labels to separately label the closed chromatin region DNA, or a portion thereof, and the open chromatin region DNA, or a portion thereof. Therefore, primer pairs for the amplifying the closed chromatin region DNA and primer pairs for amplifying the open chromatin region DNA may be added, in certain embodiments, to the same reaction during co-amplification. As used herein the term “primer” refers to a DNA molecule that is designed for use in annealing or hybridization methods that involve an amplification reaction. An amplification reaction is an in vitro reaction that amplifies template DNA to produce an amplicon. As used herein, an “amplicon” is a DNA molecule that has been synthesized using amplification techniques. A pair of primers may be used with template DNA, such as a sample of eukaryotic genomic DNA, in an amplification reaction, such as polymerase chain reaction (PCR), to produce an amplicon, where the amplicon produced would have a DNA sequence corresponding to sequence of the template DNA located between the two sites where the primers hybridized to the template. A primer is typically designed to hybridize to a complementary target DNA strand to form a hybrid between the primer and the target DNA strand. The presence of a primer is a point of recognition by a polymerase to begin extension of the primer using as a template the target DNA strand. Primer pairs refer to use of two primers binding opposite strands of a double stranded nucleotide segment for the purpose of amplifying the nucleotide segment between them. In certain embodiments, the primers may comprise an oligonucleotide barcode, or a portion thereof, to distinguish individual cells, nuclei, or samples. In particular embodiments, the primers may comprise a modification that may be used to separate open chromatin region DNA from closed chromatin region DNA following co-amplification. Oligonucleotide modifications that may be used for separating DNA molecules are known in the art, and any such oligonucleotide modification may be used according to the methods of the present disclosure. In some embodiments, the methods of the present disclosure may include pre-amplification of either closed chromatin region DNA or open chromatin region DNA prior to co-amplification. For example, pre-amplification may be utilized, in particular embodiments, to enrich open chromatin region DNA prior to co-amplification. Pre-amplification may, in some embodiments, may be performed by adding the primer pair specific for either closed chromatin region DNA or open chromatin region DNA, if either of these regions would benefit from enrichment prior to co-amplification. In particular embodiments, amplification to enrich either closed chromatin region DNA or open chromatin region DNA may be performed prior to (pre-amplification) or after (post-amplification) exponential co-amplification. In certain embodiments, the annealing temperature used during co-amplification may be favorable to closed chromatin region DNA amplification, open chromatin region DNA amplification, or to both closed chromatin region DNA and open chromatin region DNA amplification to control the total number of molecules amplified. In certain embodiments, the amount of amplified open chromatin region DNA and closed chromatin region DNA may be balanced by controlling the favored annealing temperature.

### H. Sample, Sub-Sample, or Individual Cell Labeling

In certain embodiments of the present disclosure, samples, sub-samples, or individual cells or nuclei may be labeled with a cell, nucleus, sub-sample, or sample oligonucleotide barcode prior to pooling the open chromatin region DNA and closed chromatin region DNA fragments from all cells, nuclei, samples, or sub-samples together. In some embodiments, the closed chromatin region DNA and the open chromatin region DNA from the same sample, sub-sample, cell, or nucleus may be labeled with the same set of oligonucleotide barcodes or a different set of oligonucleotide barcodes, for example, using prior knowledge of the open chromatin region DNA/closed chromatin region DNA barcode correspondence relationship. The closed chromatin region DNA and open chromatin region DNA, in some embodiments, may be labeled with a single sample, sub-sample, nucleus, or cell barcode or by multiple sample, sub-sample, or cell barcodes. In one embodiment, the open chromatin region DNA and closed chromatin region DNA may be labeled with a combination of sample, sub-sample, cell, or nucleus barcodes. The sample, sub-sample, cell, or nucleus barcode or barcodes may be added, for example, to one end of the closed chromatin region DNA or open chromatin region DNA fragments or may be added to both ends of the closed chromatin region DNA or open chromatin region DNA fragments. In particular embodiments, sample, sub-sample, cell, or nucleus barcodes may be added to the 5′ end, the 3′end, or to both the 5′ end and 3′ end of the closed chromatin region DNA or open chromatin region DNA fragments.

In some embodiments, tagmentation-based chemistry may be used to fragment the open chromatin region DNA. The open chromatin region DNA fragments may then, in particular embodiments, be labeled or barcoded using a transposome and different oligonucleotide sequences or barcodes. In certain embodiments, the oligonucleotide sequences or barcodes may be added by attaching different oligonucleotide sequences to the mosaic end sequences of the transposase, or the oligonucleotide sequences or barcodes may be added by using PCR primers that comprise the different oligonucleotide sequences, barcodes, or barcode combinations. Oligonucleotide sequences, barcodes, or barcode combinations, in some embodiments, may be added to open chromatin region DNA fragments combining the approaches of tagmentation and PCR.

In particular embodiments, tagmentation-based chemistry may also be used to fragment the closed chromatin region DNA. Similar to open chromatin region DNA labeling, in some embodiments, the closed chromatin region DNA fragments can be barcoded or labeled using a transposome with different oligonucleotide sequences or barcodes. In certain embodiments, the oligonucleotide sequences or barcodes may be added by attaching different oligonucleotide sequences to the mosaic end sequences of the transposase, or the oligonucleotide sequences or barcodes may be added by using PCR primers that comprise the different oligonucleotide sequences, barcodes, or barcode combinations. Oligonucleotide sequences, barcodes, or barcode combinations, in some embodiments, may be added to closed chromatin region DNA fragments combining the approaches of tagmentation and PCR. The closed chromatin region DNA and open chromatin region DNA libraries may be separated or distinguished after pooling the two libraries from single cells or sample materials, in some embodiments, by using the different barcodes added to each. In particular embodiments, at least one or at least two of the added oligonucleotide sequences or barcodes are different between the closed chromatin region DNA and open chromatin region DNA libraries. One of the oligonucleotide sequences, barcodes, or primers used to label the closed chromatin region DNA or the open chromatin region DNA, in some embodiments, may further comprise one or more specific modifications that can facilitate the separation of closed chromatin region DNA and open chromatin region DNA after pooling. In one embodiment, the modification may be a biotin modification. In particular embodiments, and as shown in FIGS. 1-3, the second labeling reaction may label open chromatin region DNA in addition to closed chromatin region DNA. Open chromatin region DNA may be distinguished from closed chromatin region DNA, however, because open chromatin region DNA will additionally comprise, in some embodiments, the first label introduced during the first labeling reaction, whereas closed chromatin region DNA will not additionally comprise this first label.

In certain embodiments, after the closed chromatin region DNA and the open chromatin region DNA of the same cell or sample material are labeled with cell, sample, sub-sample, or nuclei barcodes, the libraries from all reactions are pooled together. From this pool of cells, samples, sub-samples, or nuclei, in certain embodiments, a physical separation of the closed chromatin region DNA and the open chromatin region DNA may be performed. The method of separation may be based, in one embodiment, on a modification used to label either the closed chromatin region DNA or the open chromatin region DNA, on the different oligonucleotide sequences or barcodes used to distinguish the closed chromatin region DNA and the open chromatin region DNA, or by other methods known in the art able to distinguish the amplified closed chromatin region DNA and the amplified open chromatin region DNA.

### I. Construction of Closed Chromatin Region DNA and Open Chromatin Region DNA Libraries

In particular embodiments, following separation of closed chromatin region DNA and open chromatin region DNA, the amplified fragments may be used for high-throughput sequencing. In one embodiment, the PCR primers used to amplify the closed chromatin region DNA and open chromatin region DNA fragments comprise sequencing adaptors used for high-throughput sequencing. Methods and primers for high-throughput sequencing are known in the art and any such methods or primers may be used according to the methods of the present disclosure. In some embodiments, the separated closed chromatin region DNA and open chromatin region DNA amplification products may be used to prepare different sequencing libraries according to the research purpose and sequencing instrument requirements. The separated closed chromatin region DNA or open chromatin region DNA amplification products can also be, in certain embodiments, further enriched using the open chromatin region DNA or closed chromatin region DNA-specific labels added during previous steps. These enriched amplification products, in some embodiments, may then be used as the input material for high-throughput sequencing. High-throughput sequencing platforms are known in the art and any such high-throughput sequencing platform may be used according to the methods of the present disclosure. Non-limiting examples of which include next generation sequencing, single molecule sequencing, and nanopore sequencing.

In certain embodiments, the amplified closed chromatin region DNA or the amplified closed chromatin region DNA and open chromatin region DNA can be used to identify a DNA sequence variation. As used herein the term “DNA sequence variation” may refer to any variation or alteration of a DNA nucleotide sequence. Non-limiting examples of types of DNA sequence variations include copy number variations or alterations (CNV/CNA), single nucleotide polymorphisms (SNPs), mutation of one or more nucleotides, insertions and deletions (indels), short tandem repeats (STRs), translocations, inversions, structure variations (SVs), and any combinations thereof. The amplified DNA can also be used, in some embodiments, to profile targeted genes or gene panels, for probe-based target capture, for exome capture, or for other capture applications. In particular embodiments, the amplified DNA can also be used to investigate DNA rearrangements and markers, to detect frequency of mutations, or for other DNA-related applications.

The amplified open chromatin region DNA, in some embodiments, may be used to investigate or identify epigenetic modifications. As used herein the term “epigenetic modification” refers to any genetic modification that alters gene activity without altering the DNA sequence. Non-limiting examples of epigenetic modifications include any modification that affects chromatin accessibility, histone modifications (for example histone acetylation, methylation, phosphorylation, ubiquitylation, sumoylation, deamination, and proline isomerization), DNA methylation, nucleosome positioning, loss of imprinting, chromatin structure modifications, and any combinations thereof. In particular embodiments, the amplified open chromatin region DNA can be used to directly measure the effect of chromatin structure on gene transcription and the general transcription machinery, to study the regulatory function of nucleosome positioning, to study the specific functions of genomic regions related to the regulation of gene expression, to identify cell types and cell states, to investigate the role of the tumor-associated immune cells, to detect disease-linked changes in chromatin structure and transcription regulation, to analyze population-specific chromatin accessibility, to investigate the interactions between proteins and DNA, and to study the DNA binding sites of protein of interest, and to examine gene regulation and chromatin-associated protein binding.

### J. Kits

In certain aspects, the present disclosure provides kits that may be used for performing the methods provided by the present disclosure. In some embodiments, such kits may comprise one or more of the following: a first transposase, a first adaptor molecule for labeling open chromatin region DNA, a second adaptor molecule for labeling closed chromatin region DNA and open chromatin region DNA, a chromatin disruption agent for disrupting the chromatin structure of chromosomal DNA, a second transposase, a first set of primers for amplifying labeled open chromatin region DNA, a second set of primers for amplifying open chromatin region DNA and closed chromatin region DNA, dNTPs, a DNA polymerase, an RNA polymerase, a neutralization buffer, or a cell or nuclei lysis buffer. In certain embodiments, the first adaptor molecule or the second adaptor molecule further comprises a third label comprising an oligonucleotide barcode sequence. In particular embodiments, the first set of primers or the second set of primers further comprises a third label comprising an oligonucleotide barcode sequence. In one embodiment, the third label identifies the cell, sample, sub-sample, or nuclei from which the chromosomal DNA was obtained. In another embodiment, the kit may further comprise instructions for use of the kit.

The term “about” is used to indicate that a value includes the standard deviation of the mean for the device or method being employed to determine the value. The use of the term “or” in the claims is used to mean “and/or” unless explicitly indicated to refer to alternatives only or the alternatives are mutually exclusive. When used in conjunction with the word “comprising” or other open language in the claims, the words “a” and “an” denote “one or more,” unless specifically noted otherwise. The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps. Similarly, any system or method that “comprises,” “has,” or “includes” one or more components is not limited to possessing only those components and covers other unlisted components.

Other objects, features, and advantages of the present disclosure are apparent from detailed description provided herein. It should be understood, however, that the detailed description and any specific examples provided, while indicating specific embodiments of the disclosure, are given by way of illustration only, since various changes and modifications within the spirit and scope of the disclosure will become apparent to those skilled in the art from this detailed description. Any embodiment of the present disclosure may be used in combination with any other embodiment described herein.

All references herein are incorporated herein by reference in their entirety.

### EXAMPLES

The following examples are included to illustrate embodiments of the present disclosure. It should be appreciated by those of skill in the art that the techniques disclosed in the examples that follow represent techniques discovered by the inventor to function well in the practice of the disclosure. However, those of skill in the art should, in light of the present disclosure, appreciate that many changes can be made in the specific embodiments which are disclosed and still obtain a like or similar result without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### Example 1: Approximately Simultaneous Sequencing of Closed Chromatin Region DNA and Open Chromatin Region DNA in Single Cells Using a Tube or Multiwell Plate Format

Approximately 500,000 cells from the K562 human cell line were pelleted at 500×g at 4° C. and washed twice with cold phosphate buffered saline (PBS). The cell suspension was permeabilized on ice for 5 minutes using lysis buffer (10 mM Tris-HCl, 10 mM NaCl, 3 mM MgCl₂, 0.1% Tween20, 0.1% NP40, 0.01% Digitonin, 1% BSA), and then washed with 1 mL of nuclei wash buffer. The washed cells were subjected to a tagmentation reaction using Tn5 transposome (TDE1, Illumina) at 37° C. for 30 minutes. The tagmentation reaction was stopped by adding EDTA or 1% BSA. Next, single cells were separated into each tube or well using a BD FACSMelody™ Cell Sorter (1 cell/tube or 1 cell/well). Following sorting, 2 μl lysis buffer containing 30 mM Tris-HCl, pH 8.0, 0.5% Tween 20, 0.5% TritonX-100, and 1.07 mAU/μl protease was added into each tube or well. Lysis was carried out at 55° C. for 30 minutes, and then protease was inactivated at 75° C. for 15 minutes. Next, 2 μl of a tagmentation mixture containing 1.92×TD buffer (Illumina), and in-house assembled Tn5 were added to each tube or well. The second tagmentation reaction was carried out at 55° C. for 10 minutes. Notably, the in-house assembled Tn5 transposome was assembled using different sequences compared to the Illumina TDE1 transposome. The TDE1 transposome was assembled using Tn5 transposase, oligo 1: 5′-TCGTCGGC AGCGTCAGATGTGTATAAGAGACAG-3′ (SEQ ID NO:2), oligo 2: 5′-/5phos/CTG TCTCTTATACACATCT-3′ (SEQ ID NO:3), and oligo 3: 5′-GTCTCGTGGGCT CGGAGATGTGTATAAGAGACAG-3′ (SEQ ID NO:4). The in-house assembled Tn5 transposome was assembled using Tn5 transposase, oligo 4: 5′-GCCTCCCTCGC GCCATAGATGTGTATAAGAGACAG-3′ (SEQ ID NO:5), oligo 2: 5′-/5phos/CTG TCTCTTATACACATCT-3′ (SEQ ID NO:3), and oligo 5: 5′-CTTGCCAGCC CGCTCAGAGATGTGTATAAGAGACAG-3′ (SEQ ID NO:6).

Following the second tagmentation reaction, 2 μl of neutralization buffer containing 0.1 μl EDTA, 0.4 μl 10 mM dNTPs, 0.1 μl wellDA-PCR-S5XX (AATGATACGGC GACCACCGAGATCTACACNNNNNNNNGCCTCCCTCGCGCCAT (SEQ ID NO:7) 100 μM, wherein NNNNNNNN represents the cell barcode or part of the cell barcode), 0.1 μl ATAC-S5XX (AATGATACGGCGACCACCGAGATCTACACNNNNNNNNTCGTCG GCAGCGTC (SEQ ID NO:8) 100 μM, wherein NNNNNNNN represents the cell barcode or part of the cell barcode), 1 μl 5×KAPA HiFi Fidelity Buffer, and 0.3 μl H₂O was added to each tube or well. Neutralization was carried out at 50° C. for 30 minutes. Then 2 μl of PCR mixture containing 1 μl 5×KAPA HiFi Fidelity Buffer, 0.35 μl MgCl₂ (100 mM), 0.1 μl wellDA-PCR-N7XX (CAAGCAGAAGACGGCATACGAGATNNNNNNNNCTTGCCAGC CCGCTCAG (SEQ ID NO:9) 100 μM, wherein NNNNNNNN represents the cell barcode or part of the cell barcode), 0.1 μl MD_N7XX_IN (CTGAGTCGGAGACACGCANNN NNNNNGTCTCGTGGGCTCGG (SEQ ID NO:10), 100 μM, wherein NNNNNNNN represents the cell barcode or part of the cell barcode), 0.13 μl H₂O, and 0.32 μl KapKAPA HiFi HotStart DNA Polymerase (1 U/μL) was added. The amplification reaction was performed using: 72° C. for 5 minutes, 98° C. for 30 seconds, 18 cycles of 98° C. for 15 seconds, 63° C. for 30 seconds, 72° C. for 30 seconds, then 72° C. for 1 minute. The PCR product was then purified using 1.8× Ampure XP beads and eluted with 15 μl water. To further enrich the ATAC fragments from the amplification mixture, 5 μl of the above product, together with 12.5 μl 2×KAPA HiFi HotStart ReadyMix, 1 μl Bioo-PCR-F (AATGATACGGCGACC ACCGAGATCTACAC (SEQ ID NO:11) 10 μM), MD_N7XX_out (CAAGCAGAAG ACGGCATACGAGATNNNNNNNNCTGAGTCGGAGACACGCA (SEQ ID NO:12), 10 μM wherein NNNNNNNN represents the library or sample barcode), and water subjected to the following amplification reactions: 98° C. for 30 seconds, 12 cycles of 98° C. for 15 seconds, 55° C. for 30 seconds, 72° C. for 30 seconds, then 72° C. for 1 minute. The enriched amplification product was purified with 1.5× Ampure beads, and the DNA library and the enriched ATAC library were then sequenced using MiSeq (Illumina).

### Example 2: Approximately Simultaneous Sequencing of Closed Chromatin Region DNA and Open Chromatin Region DNA in Single Cells Using a Nanowell Chip Format

Approximately 500,000 cells were pelleted at 500×g at 4 C° and the washed once with cold PBS+0.04% BSA. The cell suspension was permeabilized on ice for 5 minutes using lysis buffer (10 mM Tris-HCl, 10 mM NaCl, 3 mM MgCl₂, 0.1% Tween20, 0.1% NP40, 0.01% Digitonin, 1% BSA), and then washed with 1 mL of nuclei wash buffer. The washed cells were subjected to a tagmentation reaction using loaded Tn5 transposome at 37° C. for 30 minutes. The tagmentation reaction was stopped by adding 1% BSA. The tagmented single cell suspensions were then diluted to 40,000 cells/ml with resuspension buffer containing 0.5×PBS and DAPI. Next, the diluted cell suspensions were dispensed onto a 350 nl nanowell chip (Takara) using the ICELL8 CX Single-Cell System (Takara). The chip was scanned and only nanowells containing single cells were selected for downstream experiments. Then, 35 nl lysis buffer containing 5% Tween-20, 0.5% TritonX-100, 30 mM Tris-HCL, pH 8.0, 1.36 AU/ml protease were added into each well. Lysis was carried out at 55° C. for 30 minutes and protease was inactivated at 75° C. for 15 minutes. Next, 35 nl of tagmentation mixture containing 2×TD buffer (Illumina) and 1.35 nl of Tn5 transposome (TDE1, Illumina) were added to each well. The second tagmentation reaction was carried out at 55° C. for 10 minutes. Afterwards, 35 nl of neutralization buffer containing EDTA, 5×KAPA HiFi Fidelity Buffer, dNTPs, 2.5 μM wellDA-PCR-S5XX primer, and 7.5 μM ATAC-S5XX primer was added to each well. Neutralization was carried out at 50° C. for 30 minutes. Next, 35 nl of 17 index mixture containing Mg²+, 5×KAPA HiFi Fidelity Buffer, 2.5 μM wellDA-PCR-N7XX primer, and 7.5 μM MD_N7XX_IN primer was added to each well. Finally, 35 nl PCR reaction mixture containing 5×KAPA HiFi Fidelity Buffer and KAPA HiFi HotStart DNA Polymerase (1 U/μL) was dispensed into each well. The amplification reaction was performed as follows: 72° C. for 8 minutes, 98° C. for 30 seconds, 12 cycles of 98° C. for 20 seconds, 63° C. for 30 seconds, 72° C. for 1 minute. A final elongation was performed for 2 minutes at 72° C. PCR products from all wells were pooled and then purified with Ampure XP beads. To further enrich DNA fragments from the purified product, 30 ng of the above product, together with 25 μl 2×KAPA HiFi HotStart ReadyMix, 1.5 μl Bioo-PCR-F (AATGATACGGCGACCACCGAGATCTACAC (SEQ ID NO:11) 10 μM), 1.5 μl MD_N7XX_out (CAAGCAGAAGACGGCATACGAGATNNNNNNNNCTGAGTCGGA GACACGCA, (SEQ ID NO:12), 10 μM wherein NNNNNNNN represents the library or sample barcode), and water were incubated subjected to amplification using the following conditions: 98° C. for 30 seconds, 5 cycles of 98° C. for 15 seconds, 55° C. for 30 seconds, 72° C. for 30 seconds, then 72° C. for 1 minutes. To further enrich ATAC fragments from the purified product, 30 ng of the above product, together with 25 μl 2×KAPA HiFi HotStart ReadyMix, 1.5 μl Bioo-PCR-F (10 μM), 1.5 μl Bioo-PCR-R (CAAGCAGAAGACG GCATACGAGAT (SEQ ID NO:13), and water were incubated and subjected to amplification using the following conditions: 98° C. for 30 seconds, 5 cycles of 98° C. for 10 seconds, 63° C. for 30 seconds, 72° C. for 1 minute, then 72° C. for 2 minutes. The enriched ATAC products were purified using Ampure XP beads. The enriched DNA and ATAC libraries were then sequenced using NextSeq2000 (Illumina).

### Example 3: Approximately Simultaneous Sequencing of Closed Chromatin Region DNA and Open Chromatin Region DNA in Single MDA-MB-231 Cells Using a Nanowell Chip Format

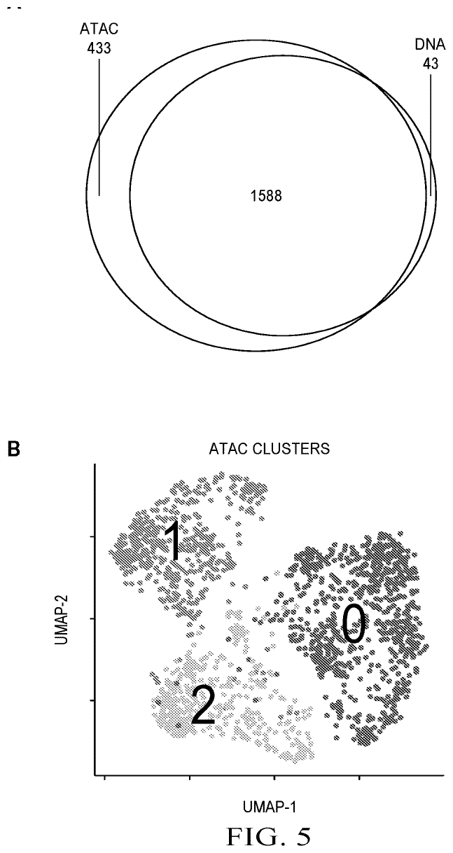

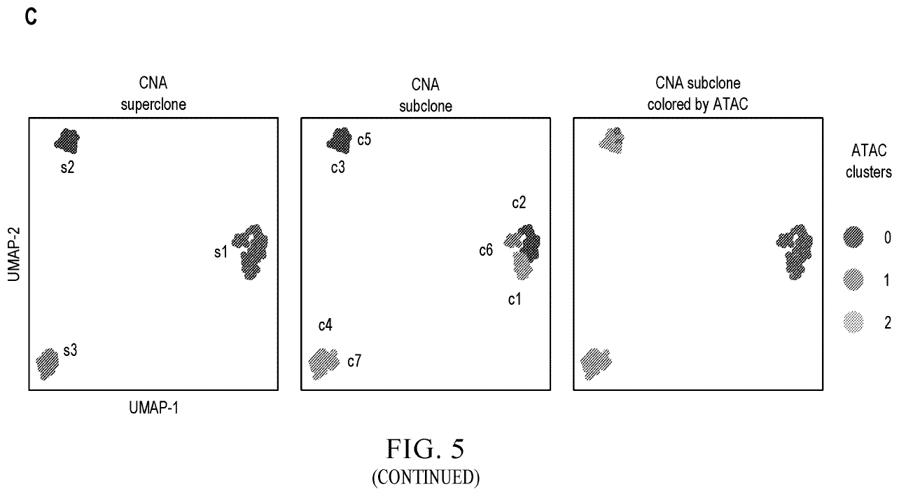

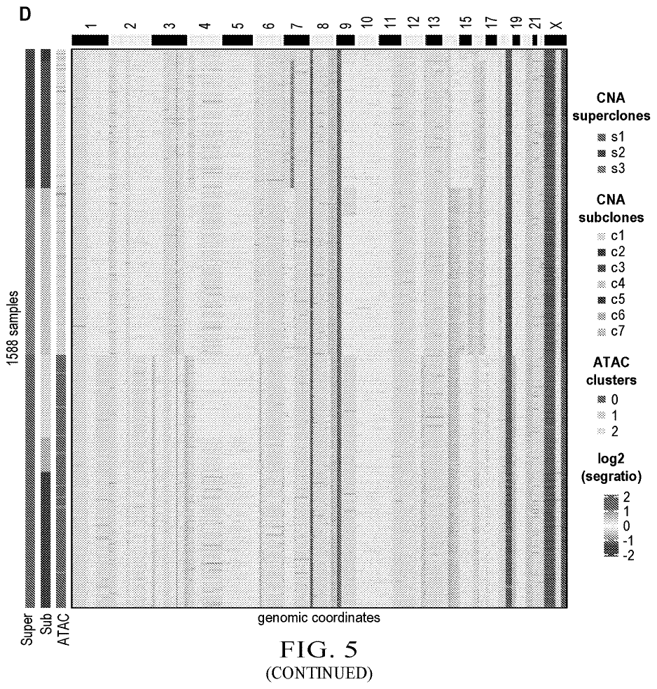

Following the protocol described above in Example 2, genomic (closed chromatin region DNA and open chromatin region DNA) and epigenomic (open chromatin region DNA) libraries were approximately simultaneously prepared from thousands of single cells in parallel from the MDA-MB-231 breast cancer cell line using nanowell chips. Single cells were dispensed onto a 5184-well nanowell chip using the ICELL8 CX Single-Cell System. 2132 single cells were selected for downstream analysis. The results of the experiment show that 2021 cells (95%) had at least 1,000 reads per cell for the epigenome data, 1631 (77%) single cells passed quality control for the genome data, and both epigenome and genome data was successfully captured for 1588 single cells (FIG. 5, Panel A). According to the chromatin accessibility profiles, the cells were clustered into three different clusters (FIG. 5, Panel B). On average, there were 16,900 (±11,200) ATAC-seq fragments and 592,000 ((±152,000) DNA fragments sequenced in each single cell. The PCR duplication rate was 18.6% (±1.4%). The single cells were clustered into three major clusters (superclones) and 7 minor clusters (subclones) based on the copy number aberration events (DNA) (FIGS. 5, Panel C and Panel D). When cells were mapped into the UMAP space of DNA clustering based on the ATAC information, the DNA and RNA modalities were very well matched in high dimensional space (FIG. 5, Panel C). These results are also similarly presented in the single cell copy number profile heatmap (FIG. 5, Panel D). Due to the high genomic resolution of the copy number data obtained in the present experiment, the results identified multiple small subclones that could not be resolved by the ATAC-seq data. The present data suggest that the epigenomic landscape corresponds to differences in subclonal genotype, validating the technical performance of the methods provided by the present disclosure.

### Example 4: Approximately Simultaneous Sequencing of Closed Chromatin Region DNA and Open Chromatin Region DNA in Single Cells Obtained from Normal Human Breast Tissue Using a Tube or Multiwell Plate Format

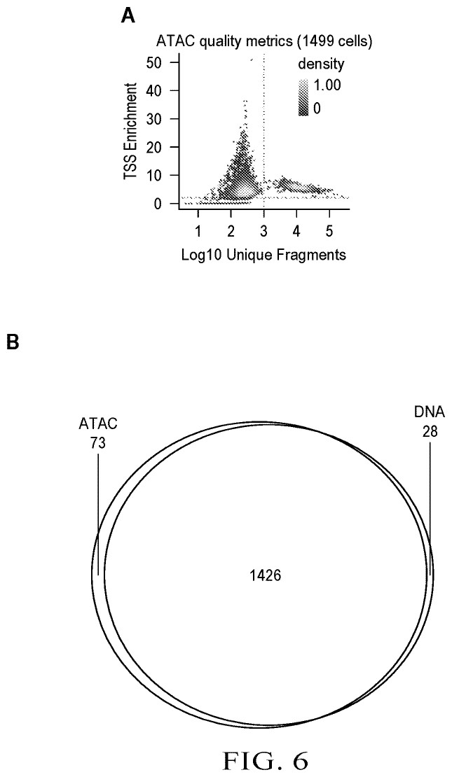

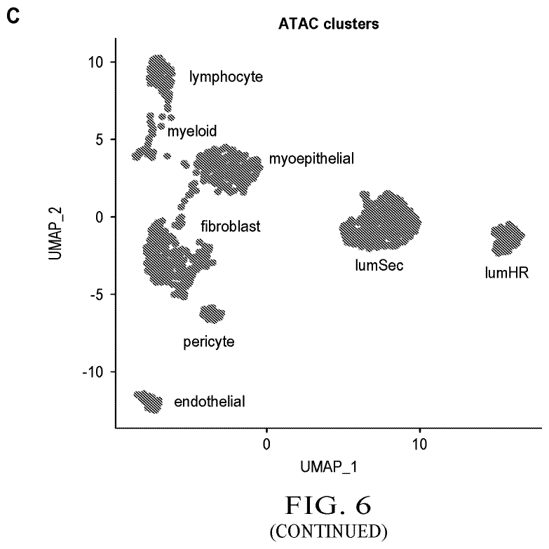

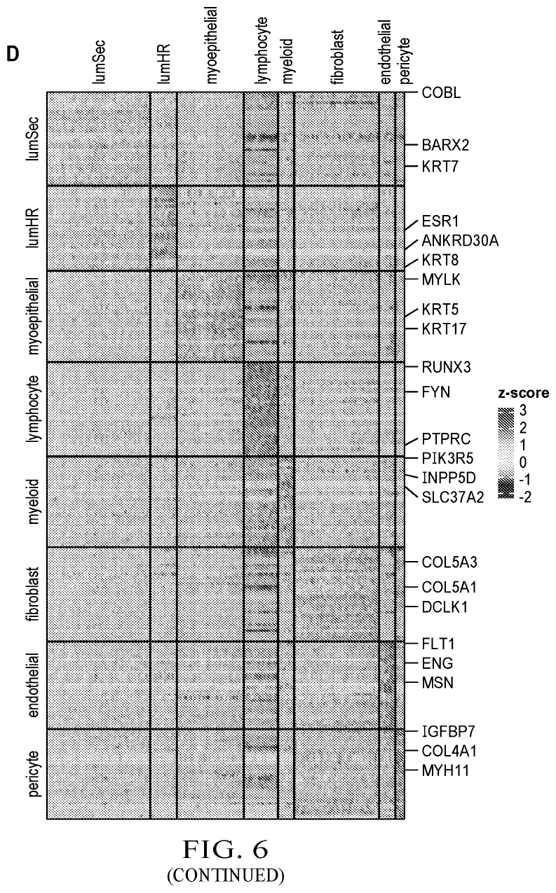

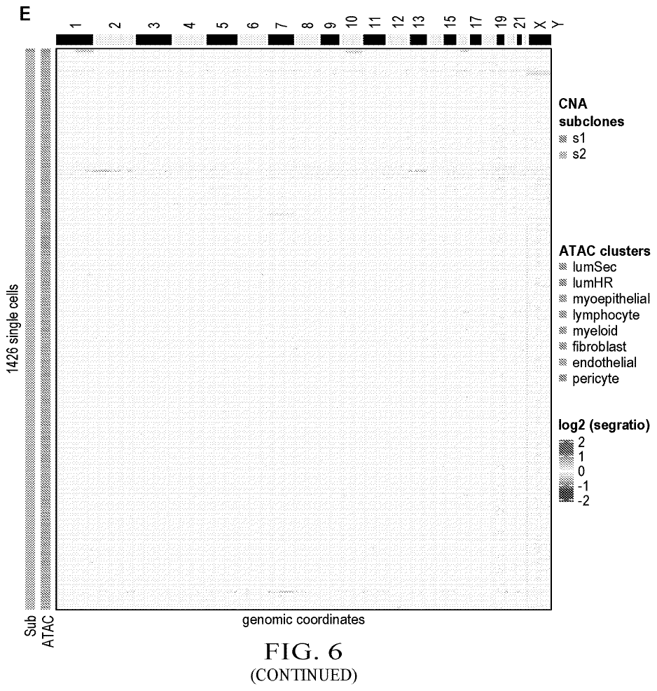

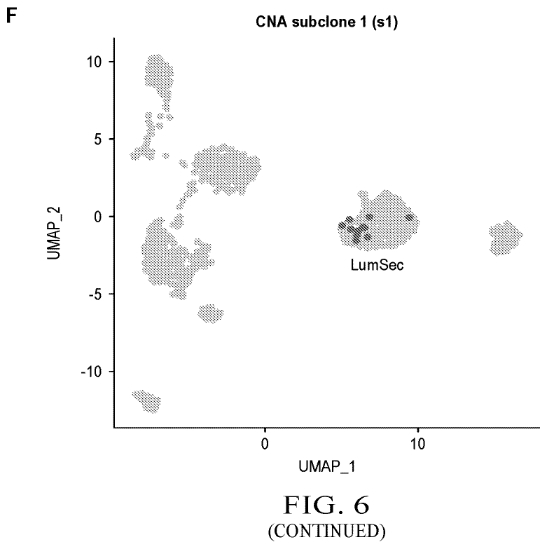

Following the protocol described above in Example 1, genomic (closed chromatin region DNA and open chromatin region DNA) and epigenomic (open chromatin region DNA) libraries were prepared from a cell suspension of dissociated human breast tissue. 1567 single cells were selected from the cell suspension for further analysis. 1499 cells (96%) passed the epigenome data quality control, having at least 1,000 fragments per cell and a TSS enrichment score (calculated by Signac) greater than 2, and 1454 cells (93%) passed the genome data quality control with at least 100,000 reads per cell (FIG. 6, Panel A). Both genome and epigenome data from 1426 single cells was successfully captured (FIG. 6, Panel B). According to the TSS-flanking scATAC signals and the inferred RNA expression, the cells were clustered into 8 different clusters (FIG. 6, Panel C), which correspond to 8 different cell types, including luminal secretory epithelial cells (lumSec) with marker genes COBL, BARX2, KRT7, luminal hormone response epithelial cells (lumHR, ESR1, ANKRD30A, KRT8), myoepithelial cells (MYKL, KRT5, KRT17), lymphocytes (RUNX3, FYN, PTPRC), myeloid cells (PIK3R5, INPP5D, SLC37A2), fibroblasts (COL5A3, COL5A1, DCLK1), endothelial cells (FLT1, ENG, MSN), and pericytes (IGFBP7, COL4A1, MYH11) (FIG. 6, Panel D). The genome copy number data did not identify any copy number variations except for in a single subclone, which comprised a chr1q amplification and a chr10q deletion. The absence of copy number variations in almost all of the cells was expected because the tissue used was normal human breast tissue comprising a diploid genome (FIG. 6, Panel E). Interestingly, all of the 12 cells in subclone 1 shared a chromosome 1 amplification with same break point, suggesting the copy number aberrations of subclone 1 represent a true biological event, rather than random technical noise. Notably, all those 12 cells were from the lumSec epithelial cell cluster according to the cell types identified by their chromatin accessibility signals (FIG. 6, Panel F). This data demonstrates that the methods of the present disclosure can consistently measure single cell DNA copy number and the chromatin accessibility landscape of single cells in human tissue samples.

### Example 5: Example Workflow for Nanowell Based Simultaneous Sequencing of Closed Chromatin Region DNA and Open Chromatin Region DNA

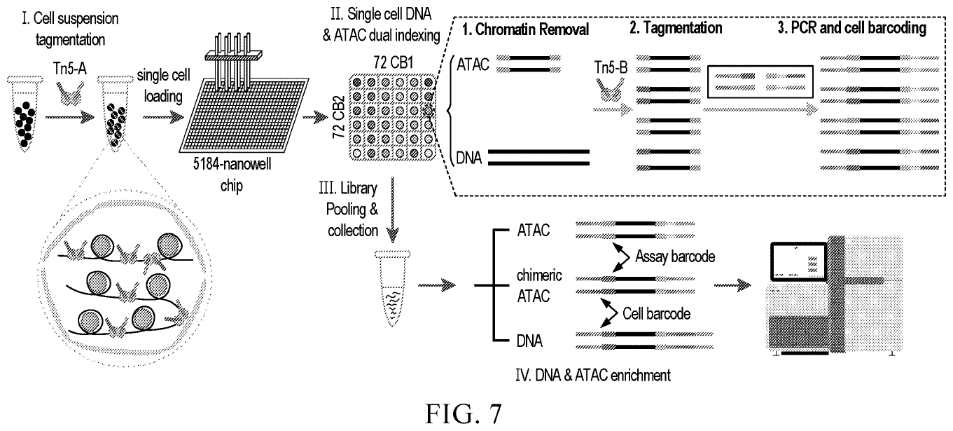

Single cell/nuclei suspensions are first tagmented with the first set of Tn5 (Tn5-A), then loaded into a nanowell chip with 5,184 nanowells. Then the chromatin is removed to expose closed DNA regions. Another set of Tn5 (Tn5-B) is used to perform tagmentation to label the closed chromatin regions, followed by PCR amplification with primers that bind to both sets of Tn5 adaptors to assign cell barcodes to each molecule. Cell barcode assigned molecules of the whole chip are then pooled together. Further, the ATAC and DNA libraries are then enriched with library specific primers to construct high throughput sequencing libraries. An overview of the workflow is shown in FIG. 7.

### Example 6: Simultaneous Sequencing of Closed Chromatin Region DNA and Open Chromatin Region DNA Close to Histone Modifications in Single Cells in the K562 Cell Line

Approximately 100,000 cells were pelleted at 500×g at 4 C° and then washed once with cold PBS+0.04% BSA. The cell suspension was permeabilized on ice for 10 minutes using Nuclear Extraction (NE) Buffer (10 mM KCl, 0.1% TritonX-100, 20% Glycerol, 0.5 mM Spermidine, 1× Protease inhibitors), and then washed with 150 μL of antibody buffer (0.1% BSA, 2 mM EDTA, 0.01% Digitonin, 150 mM NaCl, 20 mM HEPES, 0.5 mM Spermidine, 1× Protease inhibitor). The washed cells were subjected to antibody incubation overnight in 4° C. with 4% antibody (e.g., H3K4me3 Abcam, ab213224) in antibody buffer. On the second day, cells were applied to secondary antibody incubation with 2^{nd} antibody buffer (1% 2^{nd} antibody (guinea pig anti-rabbit Novus Biologicals, NBP1-72763), 0.01% Digitonin, 150 mM NaCl, 20 mM HEPES, 0.5 mM Spermidine, 1× Protease inhibitor) at room temperature. The cells were washed with digitonin buffer (0.01% Digitonin, 300 mM NaCl, 20 mM HEPES, 0.5 mM Spermidine, 1× Protease inhibitor) and then incubated with tagmentation master mix (5% pAG-Tn5, 110 mM MgCl₂, 0.01% Digitonin, 300 mM NaCl, 20 mM HEPES, 0.5 mM Spermidine, 1× Protease inhibitor) for 1 hr at 37° C. The tagmentation reaction was stopped by adding 1% BSA. The tagmented single cell suspensions were then diluted to 40,000 cells/ml with resuspension buffer containing 0.5×PBS and DAPI. Next, the diluted cell suspensions were dispensed onto a 350 nl nanowell chip (Takara) using the ICELL8 CX Single-Cell System (Takara). The chip was scanned and only nanowells containing single cells were selected for downstream experiments. Then, 35 nl lysis buffer containing 5% Tween-20, 0.5% TritonX-100, 30 mM Tris-HCL, pH 8.0, 1.36 AU/ml protease were added into each well. Lysis was carried out at 55° C. for 30 minutes and protease was inactivated at 75° C. for 15 minutes. Next, 35 nl of tagmentation mixture containing 2×TD buffer (Illumina) and 0.4 nl of Tn5 transposome (Tn5 Diagenode) were added to each well. The second tagmentation reaction was carried out at 55° C. for 9 minutes. Afterwards, 35 nl of neutralization buffer containing EDTA, 5×KAPA HiFi Fidelity Buffer, dNTPs, 2.5 μM wellDA-PCR-S5XX primer, and 7.5 μM ATAC-S5XX primer was added to each well. Neutralization was carried out at 50° C. for 30 minutes. Next, 35 nl of 17 index mixture containing Mg²+, 5×KAPA HiFi Fidelity Buffer, 2.5 μM wellDA-PCR-N7XX primer, and 7.5 μM MD_N7XX_IN primer was added to each well. Finally, 35 nl PCR reaction mixture containing 5×KAPA HiFi Fidelity Buffer and KAPA HiFi HotStart DNA Polymerase (1 U/μL) was dispensed into each well. The amplification reaction was performed as follows: 72° C. for 8 minutes, 98° C. for 30 seconds, 12 cycles of 98° C. for 20 seconds, 63° C. for 30 seconds, 72° C. for 1 minute. A final elongation was performed for 2 minutes at 72° C. PCR products from all wells were pooled and then purified with Ampure XP beads. To further enrich Cut&Tag fragments from the purified product, 30 ng of the above product, together with 25 μl 2×KAPA HiFi HotStart ReadyMix, 1.5 μl Bioo-PCR-F (10 μM), 1.5 μl Bioo-PCR-R (CAAGCAGAAGACG GCATACGAGAT (SEQ ID NO:13), and water were incubated and subjected to amplification using the following conditions: 98° C. for 30 seconds, 5 cycles of 98° C. for 10 seconds, 63° C. for 30 seconds, 72° C. for 1 minute, then 72° C. for 2 minutes. The enriched ATAC products were purified using Ampure XP beads. The mixed DNA-Cut&Tag and enriched Cut&Tag libraries were then sequenced using NextSeq2000 (Illumina).

### Example 7: Simultaneous Sequencing of DNA and Cut&Tag in Single Cells in the K562 Cell Line

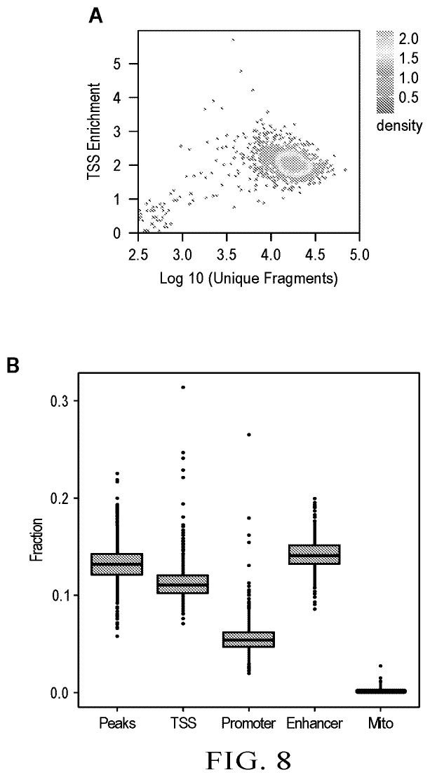

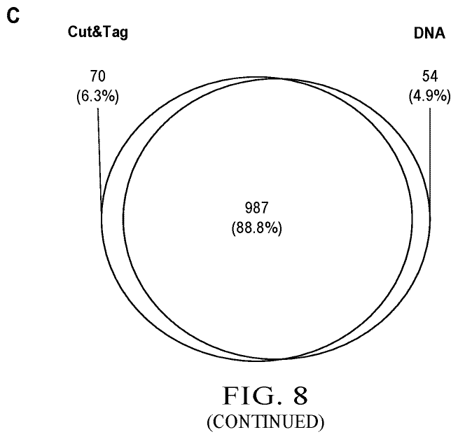

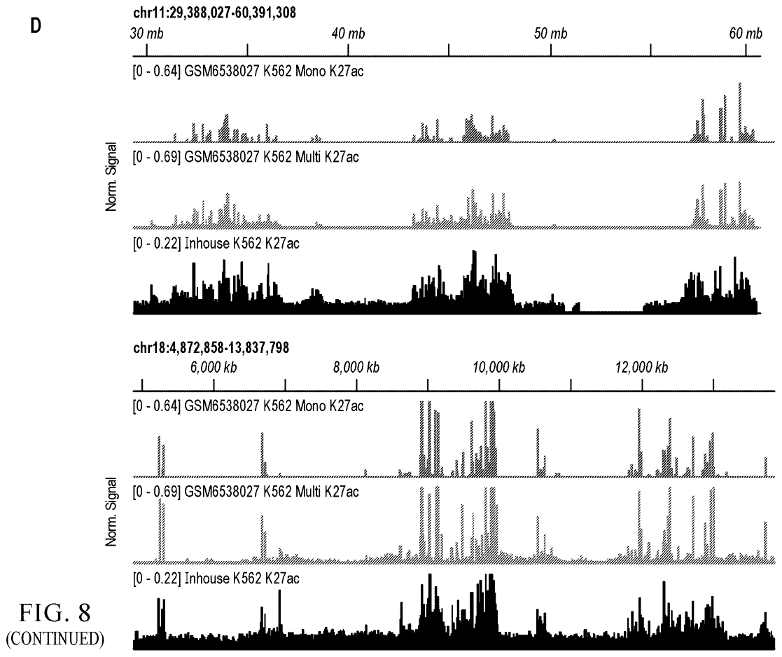

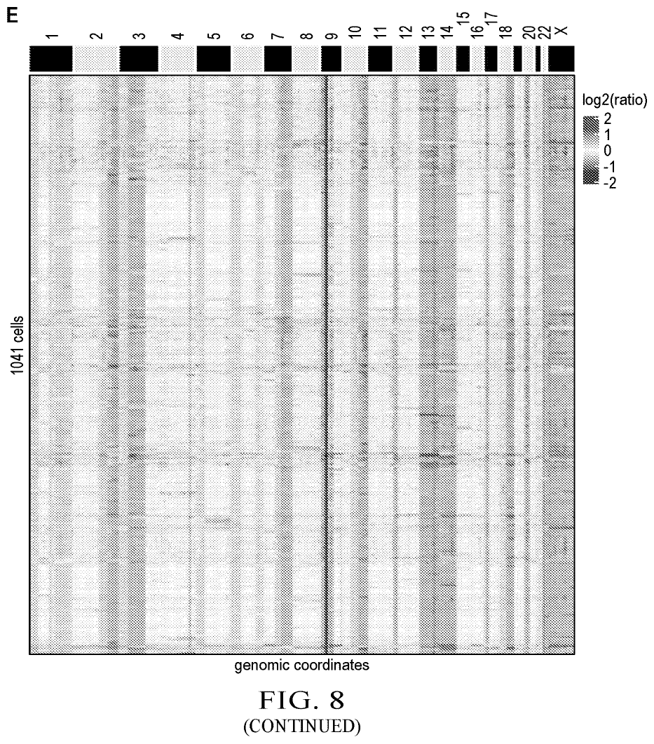

Following the protocol described above in Example 6, genomic (closed chromatin region DNA and open chromatin region DNA) and epigenomic (open chromatin region DNA in selected histone modification region) libraries were prepared from a human cell line K562. 1552 single cells were selected from the cell suspension for further analysis. 1057 cells (68%) passed the epigenome data quality control, having at least 5,000 fragments per cell and a TSS enrichment score (calculated by ArchR) greater than 2 (FIG. 8, Panel A), and 1041 cells (67%) passed the genome data quality control with at least 100,000 reads per cell (FIG. 8, Panel E). Both genome and epigenome data from 987 single cells was successfully captured (FIG. 8, Panel C). The median fragments overlapping peaks is 13.1%, overlapping TSS is 11%, and overlapping enhancers is 14.1% across cells, indicating good overall quality of epigenome signal captured (FIG. 8, Panel B). The H3K27Ac peaks called from the epigenome data are aligned and consistent with the previous study using a single cell approach (FIG. 8, Panel D). The genomic copy number sequencing also showed high data quality, with median 40 bin counts, 17.29% PCR duplication. The captured CNA events were consistent from previous report such as chr1q gain, chr3q loss and chr 7q gain (FIG. 8, Panel E). This data demonstrates that the methods of the present disclosure can consistently measure single cell DNA copy number and the chromatin accessibility in selected histone modification region at single cell resolution.

### Example 8: Performance Evaluation of WellDA-Seq in the MDA-MB-231 Cell Line

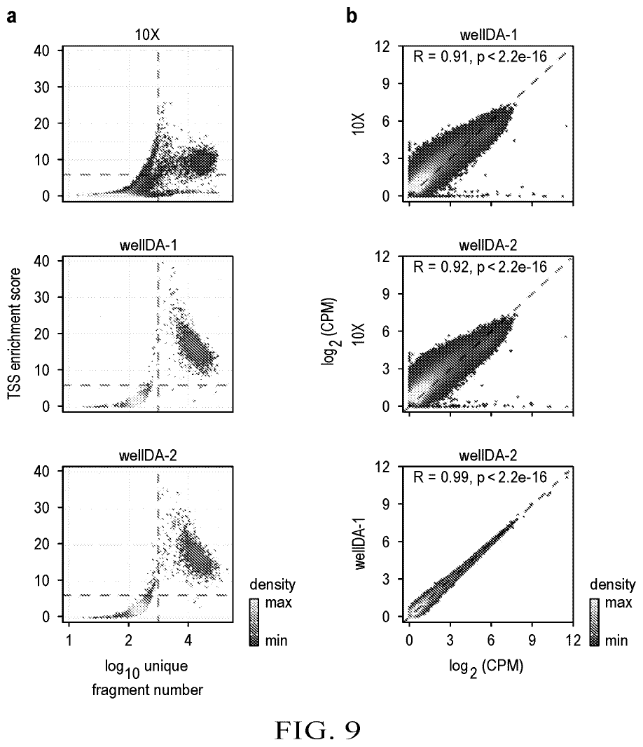

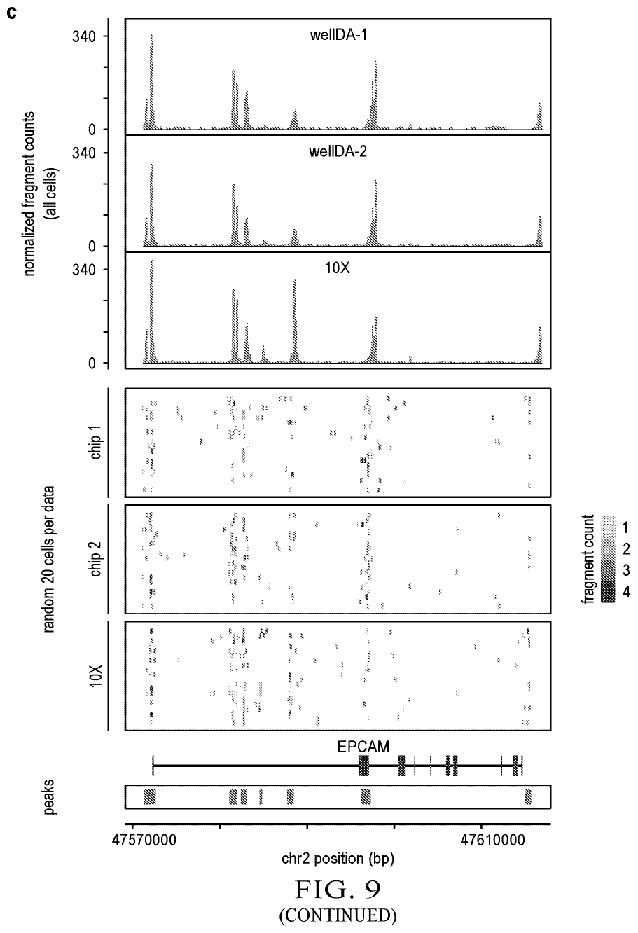

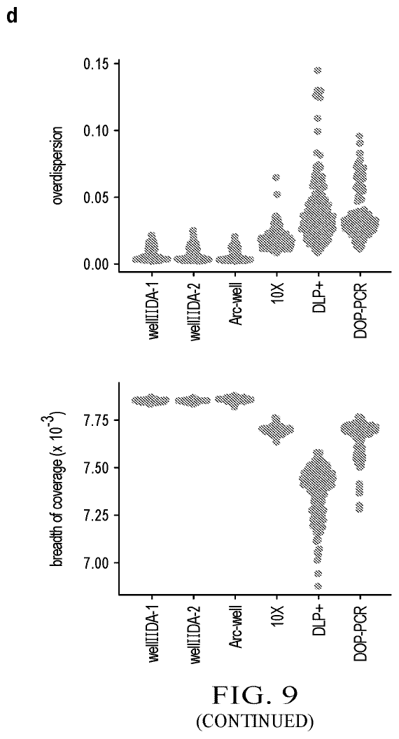

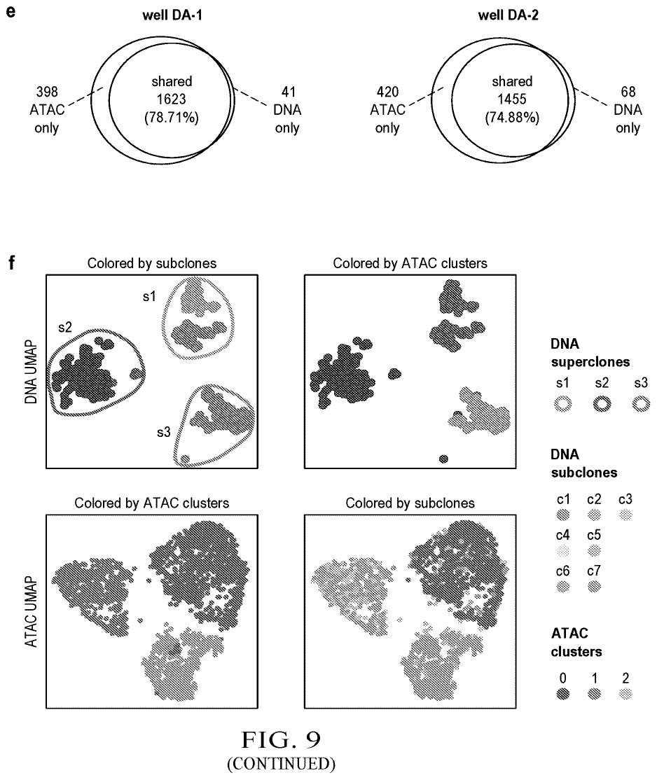

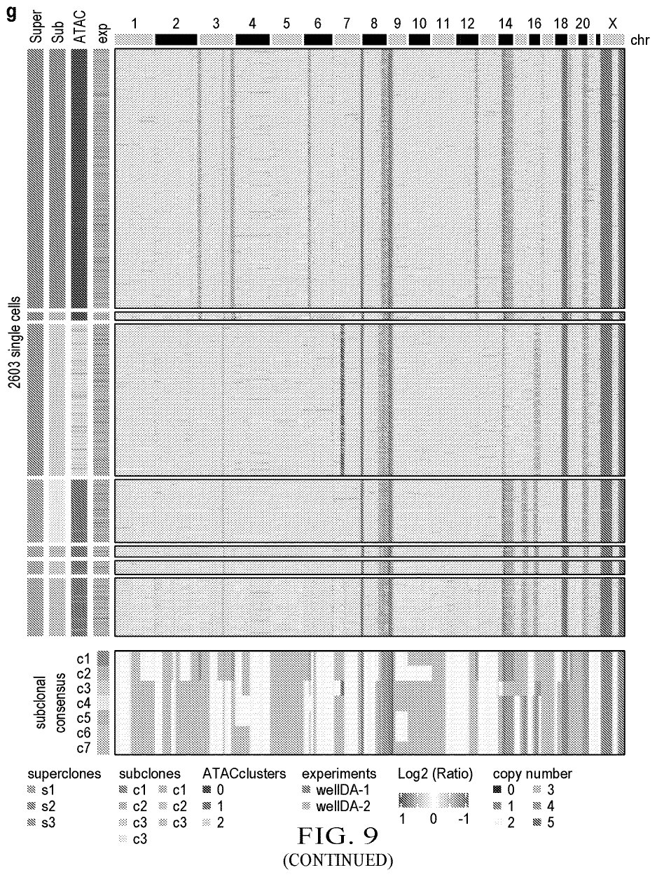

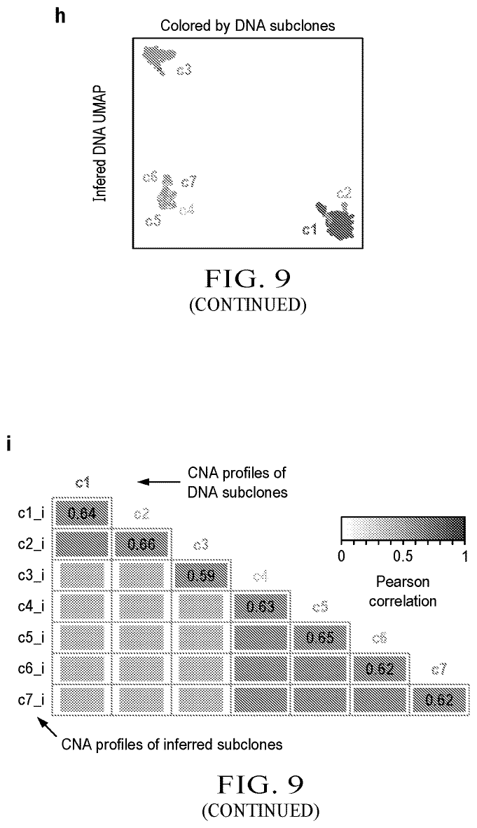

For the wellDA-seq experiments, simultaneous sequencing of closed chromatin region DNA and open chromatin region DNA was generally performed as described in Example 5. For the 10× Genomics experiments sequencing was generally performed as described in the manufacturer's instructions for Chromium Single Cell ATAC, 10× Genomics. See, for example, https://www.10xgenomics.com/products/single-cell-atac. Quality control plots for the 10× (10× Genomics) and the two wellDA-seq experiments (wellDA-1 and wellDA-2) are shown in FIG. 9, Panel A. Each dot represents one well. Comparison of the aggregated counts per million (CPM) fragments within peaks for the MDA-MB-231 cell line in the 10×, wellDA-1, and wellDA-2 experiments by Pearson correlation coefficient R and p value (p) are shown in FIG. 9, Panel B. Comparison of scATAC profiles from the 10×, wellDA-1, and wellDA-2 experiments in a region of chromosome 2 are shown in FIG. 9, Panel C. Upper panels show the aggregated profiles of all cells, and lower panels show fragments present in each of the 20 random cells. Comparison of overdispersion metrics for the genomic bin counts and breadth of coverage metrics for wellDA-seq and four other scDNA-seq methods are shown in FIG. 9, Panel D. Coverage was calculated from 120 randomly sampled cells per method and using 750K reads per cell as input. The number of cells with DNA and/or ATAC data that were profiled by the two wellDA-seq experiments is shown if FIG. 9, Panel E. UMAP showing the clustering results of single cell DNA copy number profiles from wellDA-seq, colored by DNA subclones or ATAC clusters is shown in FIG. 9, Panel F (top panel). UMAP showing the clustering results of single cell ATAC-seq profiles from wellDA-seq, colored by ATAC clusters or DNA subclones is shown in FIG. 9, Panel F (bottom panel). FIG. 9, Panel G shows single cell copy number heatmaps from two merged wellDA-seq experiments. UMAP showing the inferred single cell DNA copy number profiles from single cell ATAC-seq data of wellDA-seq is shown in FIG. 9, Panel H. Pearson correlation of single cell DNA copy number profiles from the DNA modality of wellDA-seq and the inferred single cell DNA copy number profiles from the ATAC modality are shown in FIG. 9, Panel I.

### Example 9: Simultaneously Profiling of the Single Cell Copy Number and Single Cell ATAC-seq from Two Normal Human Breast Tissues Using WellDA-seq

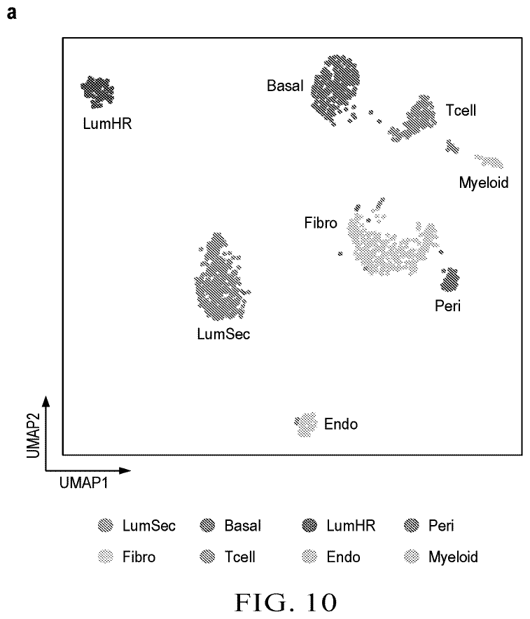

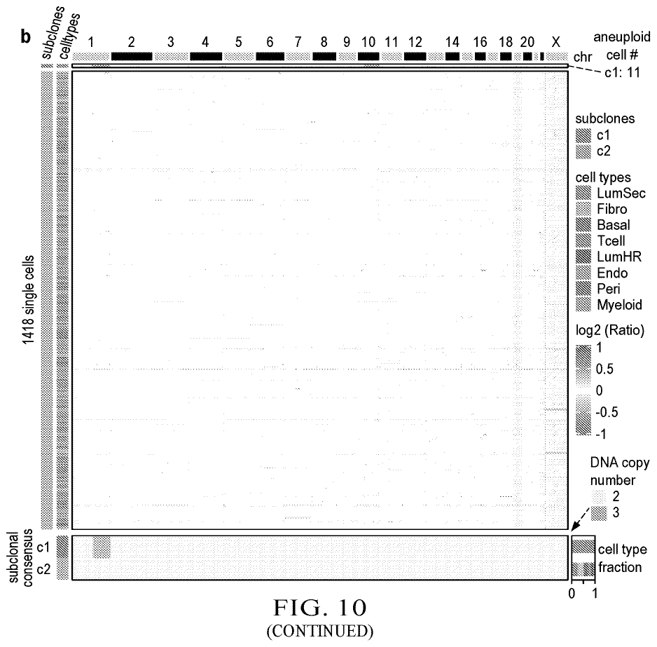

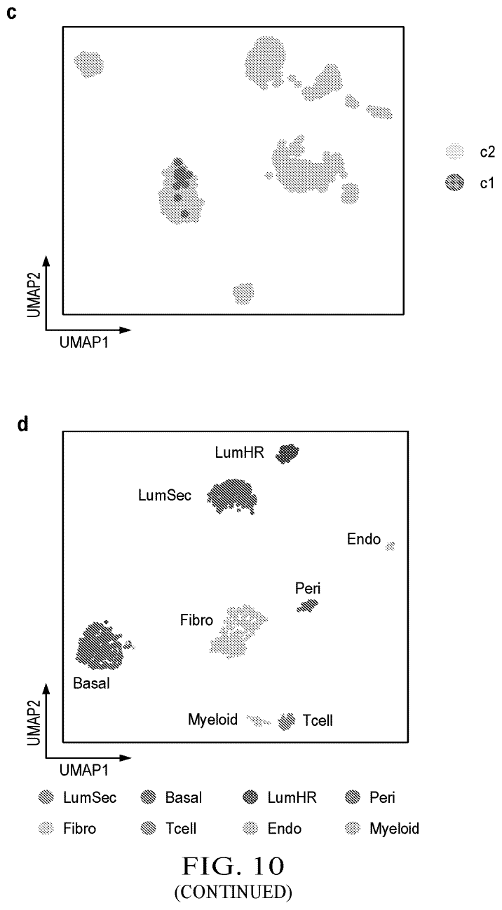

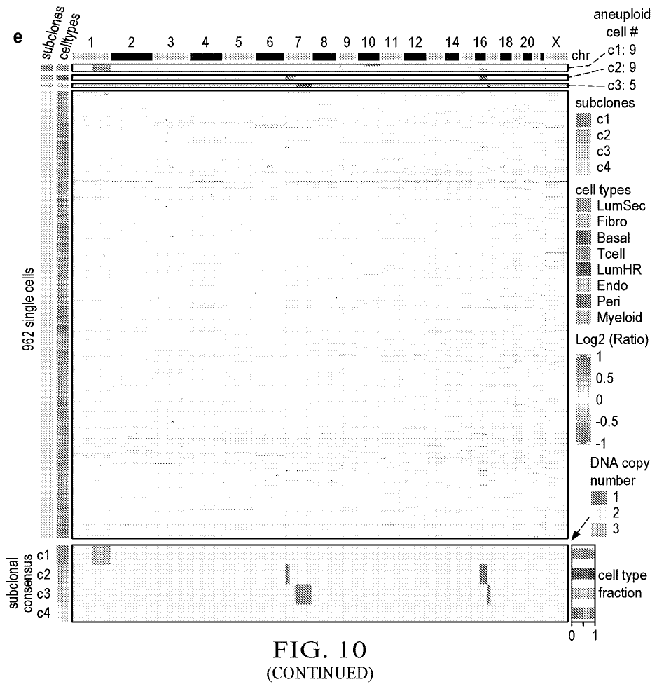

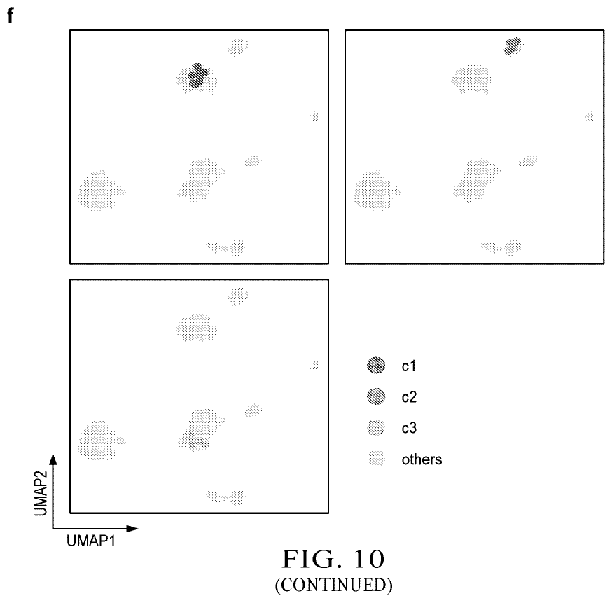

WellDA-seq was generally performed as described in Example 5. FIG. 10, Panel A shows a UMAP of scATAC-seq data of wellDA-seq from the first normal breast tissue, which identified 8 different cell types. FIG. 10, Panel B shows a heatmap of single cell DNA copy number profiles of wellDA-seq, in which 1418 single cells were profiled. The bottom panel shows the integer copy number of two different subclones identified by the singe cell DNA data. FIG. 10, Panel C shows that the aneuploid cells from subclone c1 map to the UMAP of scATAC-seq, which identified LumSec as the cell population harboring the somatic CNA events. FIG. 10, Panel D shows a UMAP of scATAC-seq data of wellDA-seq for the 2nd normal breast tissue. FIG. 10, Panel E shows a heatmap of single cell DNA copy number profiles of wellDA-seq, in which 962 single cells were profiled. FIG. 10, Panel F shows the aneuploid cells from subclone c1-c3 map to the UMAP of scATAC-seq, which identified LumSec, LumHR and fibroblast as the cell populations that harbor different somatic CNA events.

### Example 10: Overview of Single Cell ATAC Data and scDNA Data of WellDA-seq Profiled from 9 Breast Cancer Patients

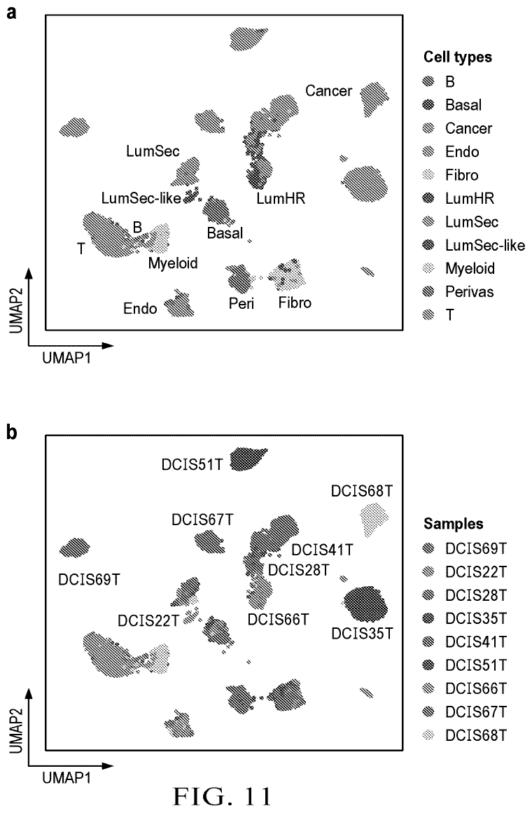

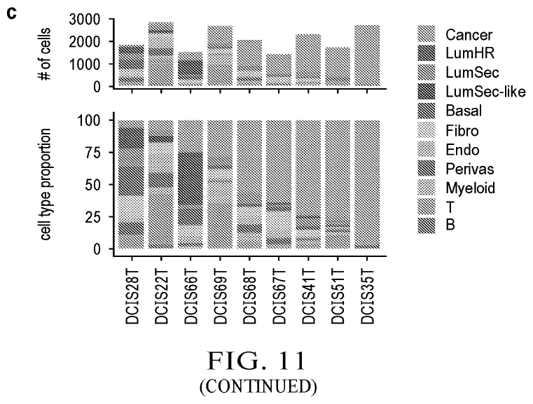

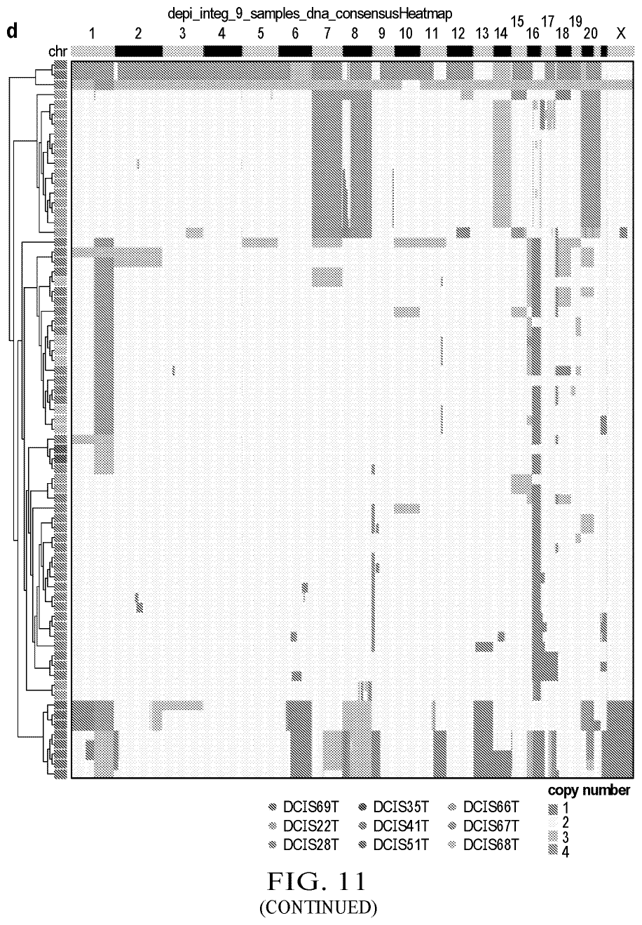

WellDA-seq was generally performed as described in Example 5. FIG. 11, Panel A shows an integrated UMAP of scATAC profiles from 9 breast cancer patients, colored by cell types. In total, 11 cell types from 19,334 cells were identified. FIG. 11, Panel B shows an integrated UMAP of scATAC profiles from 9 breast cancer patients, colored by patient. FIG. 11, Panel C shows the cell number and proportion of each cell type in each patient. FIG. 11, Panel D shows the consensus integer copy number profiles of 73 subclones identified by wellDA-seq from the 9 patients.

### Example 11: Gene Dosage Effects of Subclonal Copy Number Alteration (CNA) Events on Chromatin Accessibility

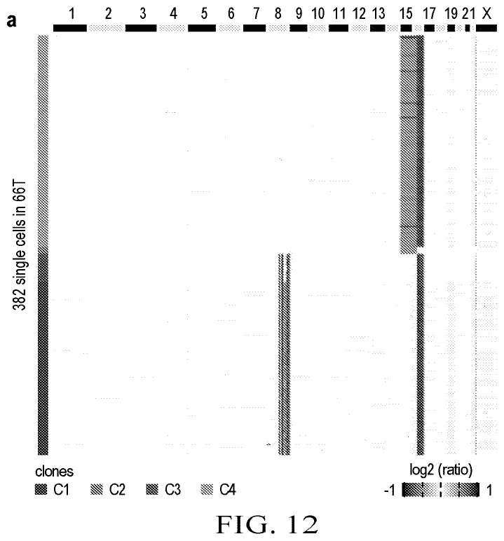

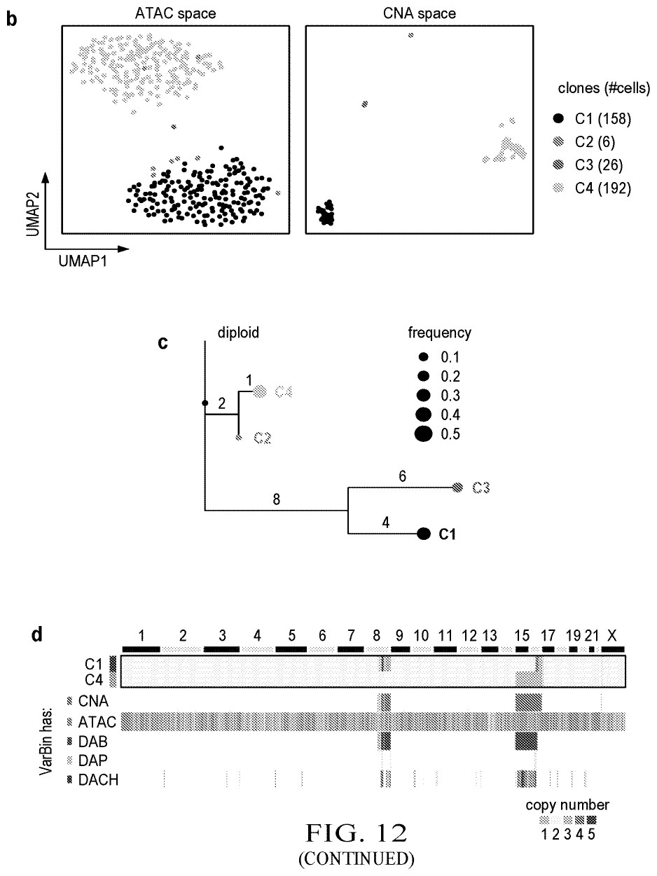

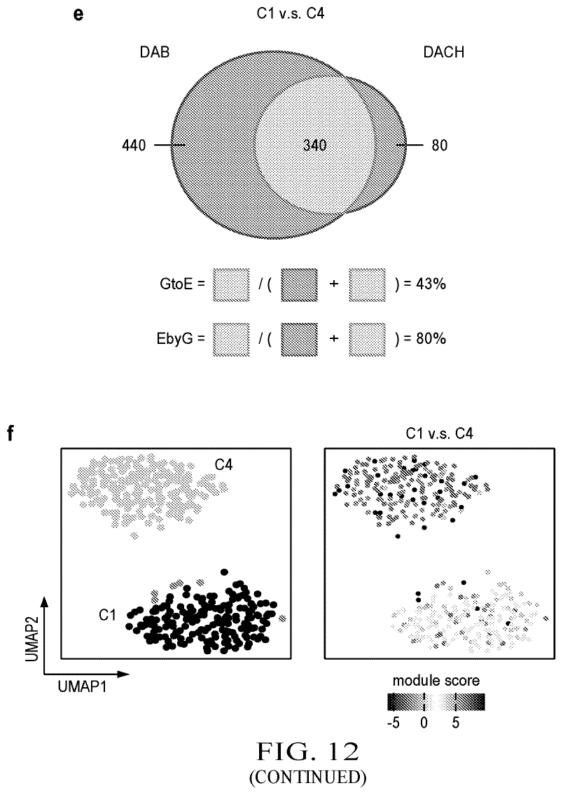

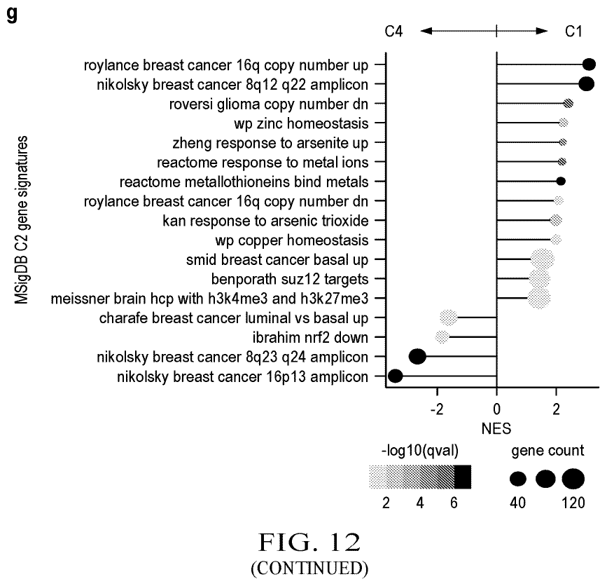

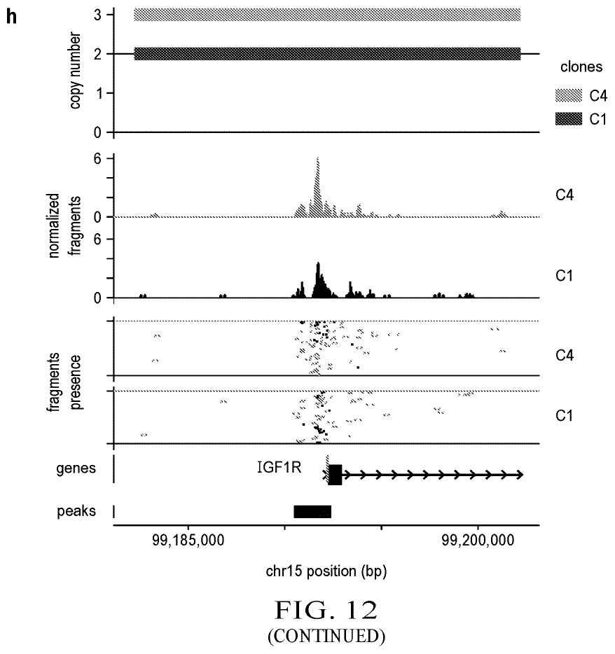

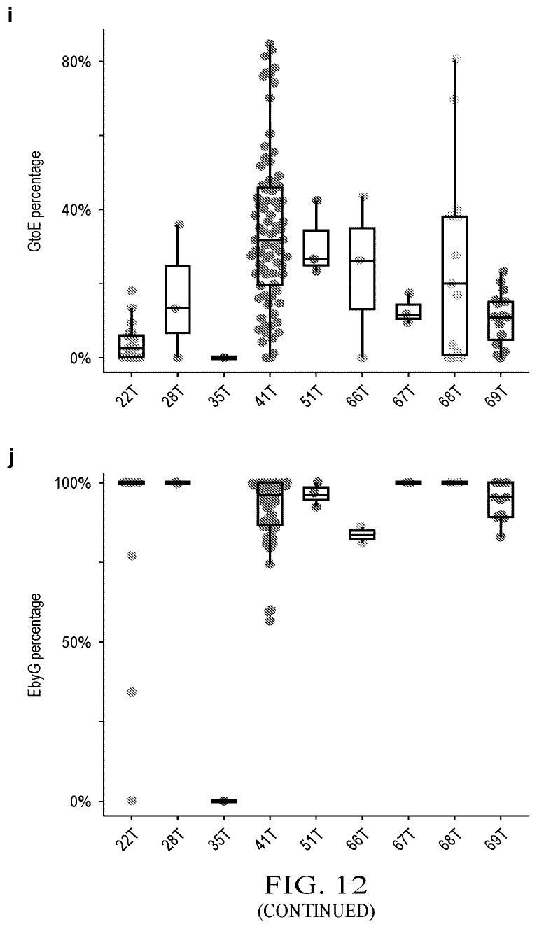

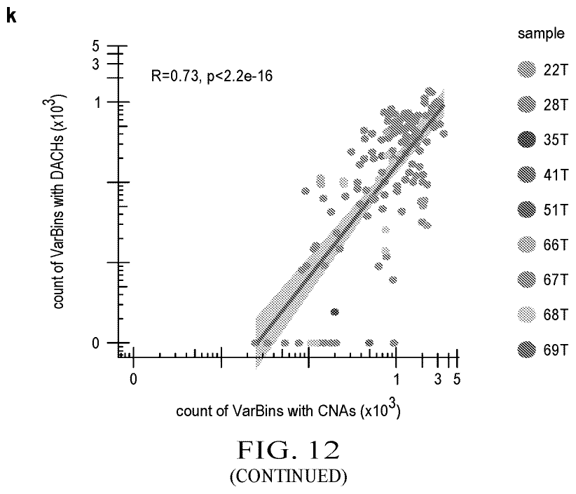

WellDA-seq was generally performed as described in Example 5. FIG. 12, Panel A shows a heatmap showing the single-cell CNA profiles of the representative sample 66T. Color bars denote the clones. FIG. 12, Panel B shows a UMAP showing the single cells based on the ATAC (left panel) and CNA (right panel) profiles. Dots (cells) are colored by the clones labeled by the cell counts. FIG. 12, Panel C shows a minimum evolution tree constructed by MEDIC2. Size of nodes denotes the frequency of cells of clones. FIG. 12, Panel D shows a heatmap showing the consensus integer CNAs of two presentative clones, C1 and C4, along genomic Varbins (columns). The bar annotations show if a genomic Varbin has a CNA event (CNA), a peak with open chromatin (ATAC), a differentially aneuploid bin (DAB), a differentially accessible ATAC peak (DAP), and a differentially accessible chromatin hub (DACH). FIG. 12, Panel E shows a Venn diagram (top panel) showing the DABs and DACHs in a comparison of the clone C1 and C4. The bottom panel shows a diagram showing the scheme of calculating GtoE and EbyG percentages. FIG. 12, Panel F shows a UMAP of the single-cell ATAC profiles that are colored by the clones (left panel) and the module scores of the DACHs in a comparison of C1 and C4. FIG. 12, Panel G shows a lollipop plot showing the significant gene signatures that were enriched for the DACHs in a comparison of C1 and C4. FIG. 12, Panel H shows a track plot at a 200 kb window of the TSS of the gene IGF1R showing the copy number (top panel), the normalized ATAC fragments (second panel), and the presence of Tn5 insertions in randomly selected cells (third panel), and the genes and peaks (bottom panel). FIG. 12, Panel I shows a boxplot showing the GtoE percentage of the samples. FIG. 12, Panel J shows a boxplot showing the EbyG percentage in the samples. FIG. 12, Panel K shows a scatter plot comparing the count of Varbins with DACHs and DABs. A Pearson correlation coefficient was calculated, and the p-value is labeled. In FIG. 12, Panels I, J, K, each dot denotes a clone-to-clone comparison.

All of the methods disclosed and claimed herein can be made and executed without undue experimentation in light of the present disclosure. While the compositions and methods of this invention have been described in terms of preferred embodiments or aspects, it will be apparent to those of skill in the art that variations may be applied to the methods and in the steps or in the sequence of steps of the method described herein without departing from the concept, spirit, and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

We claim:

1. A method of producing a DNA library for analysis of open chromatin region DNA and closed chromatin region DNA approximately simultaneously, the method comprising:
a) obtaining a sample comprising chromosomal DNA;
b) performing a first labeling reaction on said chromosomal DNA to label at least a first portion of open chromatin region DNA comprised within said chromosomal DNA with a first label;
c) disrupting the structure of said chromosomal DNA to expose at least a first portion of closed chromatin region DNA comprised within said chromosomal DNA;
d) performing a second labeling reaction on said chromosomal DNA to label:
i) at least said first portion of said exposed closed chromatin region DNA with a second label; and
ii) at least said first portion, or a second portion, of open chromatin region DNA with the second label; and
e) performing an amplification reaction to amplify said first labeled open chromatin region DNA, said second labeled exposed closed chromatin region DNA, and said second labeled open chromatin region DNA comprised within said chromosomal DNA.

2. The method of claim 1, wherein said sample comprises chromosomal DNA obtained from about 1 cell to about 1,000,000,000 cells.

3. The method of claim 2, wherein said sample comprises chromosomal DNA obtained from a single cell.

4. The method of claim 1, wherein said amplification reaction amplifies the first labeled open chromatin region DNA, the second labeled exposed closed chromatin region DNA, and the second labeled open chromatin region DNA approximately simultaneously.

5. The method of claim 1, wherein the first labeling reaction or the second labeling reaction is performed by an insertional enzyme complex.

6. The method of claim 5, wherein the insertional enzyme complex comprises a transposase.

7. The method of claim 1, the method further comprising detecting at least one DNA sequence variation in the amplified second labeled exposed closed chromatin region DNA.

8. The method of claim 7, wherein the at least one DNA sequence variation is selected from the group consisting of a mutation, a copy number alteration, a structural variation, and a DNA modification.

9. The method of claim 1, the method further comprising performing an Assay for Transposase-Accessible Chromatin (ATAC) analysis on the open chromatin region DNA to measure chromatin accessibility differences in the chromosomal DNA.

10. The method of claim 1, the method further comprising performing a Cleavage Under Targets and Tagmentation (Cut&Tag) analysis of the open chromatin region DNA to measure protein:DNA interactions of the chromosomal DNA.

11. The method of claim 10, the method further comprising detecting at least one epigenetic modification in close proximity to the open chromatin region DNA.

12. The method of claim 1, the method further comprising performing a third labeling reaction on said chromosomal DNA to label the chromosomal DNA of the sample with a third label.

13. The method of claim 12, wherein:
a) the third labeling reaction and the first labeling reaction are performed approximately simultaneously;
b) the third labeling reaction and the second labeling reaction are performed approximately simultaneously;
c) the third labeling reaction is performed before or after the first labeling reaction;
d) the third labeling reaction is performed before or after the second labeling reaction; or
e) the third labeling reaction and the amplification reaction are performed approximately simultaneously.

14. The method of claim 12, wherein:
a) the first label, the second label, or the third label is a nucleotide barcode label;
b) said first label and said second label are different;
c) said first label and said third label are different; or
d) said second label and said third label are different.

15. The method of claim 1, wherein the sample comprises a plurality of cells, and wherein the method further comprises separating the sample into a plurality of sub-samples, each comprising a single cell.

16. The method of claim 15, wherein said separating is performed prior to disrupting the structure of said chromosomal DNA.

17. The method of claim 15, the method further comprising performing a third labeling reaction on said chromosomal DNA to label the chromosomal DNA of at least one sub-sample with a third label.

18. The method of claim 15, the method further comprising performing a third labeling reaction on said chromosomal DNA to label the chromosomal DNA of each sub-sample with a unique third label.

19. The method of claim 1, further comprising separating the first labeled open chromatin region DNA from the second labeled exposed closed chromatin region DNA and the second labeled open chromatin region DNA following said step (e).

20. The method of claim 1, further comprising performing a DNA sequencing reaction to obtain the DNA sequence of the first labeled open chromatin region DNA, the second labeled exposed closed chromatin region DNA, or the second labeled open chromatin region DNA.

21. A DNA library produced by the method of claim 1.

22. A method of producing a DNA library for analysis of open chromatin region DNA and closed chromatin region DNA approximately simultaneously, the method comprising:
a) obtaining a plurality of samples comprising chromosomal DNA;
b) performing a first labeling reaction on said chromosomal DNA of each sample to label at least a first portion of open chromatin region DNA comprised within said chromosomal DNA with a first label;
c) disrupting the structure of said chromosomal DNA of each sample to expose at least a first portion of closed chromatin region DNA comprised within said chromosomal DNA;
d) performing a second labeling reaction to label:
i) at least said first portion of said exposed closed chromatin region DNA with a second label; and
ii) at least said first portion, or a second portion, of open chromatin region DNA with the second label;
e) combining the first labeled open chromatin region DNA, the second labeled exposed closed chromatin region DNA, and the second labeled open chromatin region DNA from each sample of the plurality of samples; and
f) performing an amplification reaction to amplify the first labeled open chromatin region DNA, the second labeled exposed closed chromatin region DNA, and the second labeled open chromatin region DNA comprised within said chromosomal DNA.

23. The method of claim 22, the method further comprising performing a third labeling reaction on said chromosomal DNA of each sample to label the chromosomal DNA of the sample with a unique third label, wherein said third labeling reaction is performed prior to said combining the first labeled open chromatin region DNA, the second labeled exposed closed chromatin region DNA, and the second labeled open chromatin region DNA from each sample of the plurality of samples.

24. The method of claim 22, wherein each sample of said plurality of samples comprises chromosomal DNA obtained from about 1 cell to about 1,000,000,000 cells.

25. The method of claim 24, wherein each sample of said plurality of samples comprises chromosomal DNA obtained from a single cell.

26. The method of claim 22, wherein said amplification reaction amplifies the first labeled open chromatin region DNA, the second labeled exposed closed chromatin region DNA, and the second labeled open chromatin region DNA approximately simultaneously.

27. The method of claim 23, wherein:
a) the third labeling reaction and the first labeling reaction are performed approximately simultaneously;
b) the third labeling reaction and the second labeling reaction are performed approximately simultaneously;
c) the third labeling reaction is performed before or after the first labeling reaction;
d) the third labeling reaction is performed before or after the second labeling reaction; or
e) the third labeling reaction and the amplification reaction are performed approximately simultaneously.

28. The method of claim 22, wherein at least one sample of the plurality of samples comprises a plurality of cells, and wherein the method further comprises separating the at least one sample into a plurality of sub-samples, each comprising a single cell.

29. The method of claim 28, wherein said separating is performed prior to disrupting the structure of said chromosomal DNA.

30. The method of claim 22, further comprising separating the first labeled open chromatin region DNA from the second labeled exposed closed chromatin region DNA and the second labeled open chromatin region DNA following said step (f).

31. The method of claim 22, further comprising performing a DNA sequencing reaction to obtain the DNA sequence of the first labeled open chromatin region DNA, the second labeled exposed closed chromatin region DNA, or the second labeled open chromatin region DNA following said step (f).

32. A DNA library produced by the method of claim 22.

33. A method of identifying a DNA sequence variation or an epigenetic modification in a sample, the method comprising:
a) obtaining a sample comprising chromosomal DNA;
b) performing a first labeling reaction on said chromosomal DNA to label at least a first portion of open chromatin region DNA comprised within said chromosomal DNA with a first label;
c) disrupting the structure of said chromosomal DNA to expose at least a first portion of closed chromatin region DNA comprised within said chromosomal DNA;
d) performing a second labeling reaction to label:
i) at least said first portion of said exposed closed chromatin region DNA with a second label; and
ii) at least said first portion, or a second portion, of open chromatin region DNA with the second label;
e) performing an amplification reaction to amplify the first labeled open chromatin region DNA, the second labeled exposed closed chromatin region DNA, and the second labeled open chromatin region DNA comprised within said chromosomal DNA;
f) performing a DNA sequencing reaction to obtain the DNA sequence of the first labeled open chromatin region DNA, the second labeled exposed closed chromatin region DNA, or the second labeled open chromatin region DNA comprised within said chromosomal DNA; and
g) identifying said DNA sequence variation or said epigenetic modification in said sample.

34. The method of claim 33, wherein said sample comprises chromosomal DNA obtained from about 1 cell to about 1,000,000,000 cells.

35. The method of claim 34, wherein said sample comprises chromosomal DNA obtained from a single cell.

36. The method of claim 33, wherein the sample comprises a plurality of cells, and wherein the method further comprises separating the sample into a plurality of sub-samples, each comprising a single cell.

37. The method of claim 36, wherein said separating is performed prior to disrupting the structure of said chromosomal DNA.

38. The method of claim 33, wherein said amplification reaction amplifies the first labeled open chromatin region DNA, the second labeled exposed closed chromatin region DNA, and the second labeled open chromatin region DNA approximately simultaneously.

39. The method of claim 33, the method comprising identifying said DNA sequence variation in the amplified second labeled exposed closed chromatin region DNA.

40. The method of claim 33, wherein the DNA sequence variation is selected from the group consisting of a mutation, a copy number alteration, a structural variation, and a DNA modification.

41. The method of claim 33, wherein identifying the epigenetic modification comprises performing a Cleavage Under Targets and Tagmentation (Cut&Tag) analysis of the open chromatin region DNA to measure protein:DNA interactions.

42. The method of claim 41, the method further comprising detecting at least one epigenetic modification in close proximity to the open chromatin region DNA.

43. The method of claim 33, wherein identifying the epigenetic modification comprises performing an Assay for Transposase-Accessible Chromatin (ATAC) analysis on the open chromatin region DNA to measure chromatin accessibility differences in the chromosomal DNA.

44. The method of claim 33, further comprising separating the first labeled open chromatin region DNA from the second labeled exposed closed chromatin region DNA and the second labeled open chromatin region DNA following said step (f).

45. The method of claim 33, wherein said DNA sequence variation or epigenetic modification is associated with a condition selected from the group consisting of cancer, a genetic disease or condition, a developmental disease or condition, or an immunological disease or condition.

46. A kit comprising:
a) a first transposase;
b) a first adaptor molecule for labeling open chromatin region DNA;
c) a second adaptor molecule for labeling closed chromatin region DNA and open chromatin region DNA; and
d) a chromatin disruption agent for disrupting the chromatin structure of chromosomal DNA.

47. The kit of claim 46, wherein said kit further comprises a second transposase.

48. The kit of claim 46, wherein said kit further comprises a first set of primers for amplifying labeled open chromatin region DNA and a second set of primers for amplifying open chromatin region DNA and closed chromatin region DNA.

49. The kit of claim 46, wherein the first adaptor molecule or the second adaptor molecule further comprises a third label comprising an oligonucleotide barcode sequence.

50. The kit of claim 48, wherein the first set of primers or the second set of primers further comprises a third label comprising an oligonucleotide barcode sequence.


SEQUENCE LISTING (13 sequences)

SEQ ID NO: 1 - DNA, 19 bp, synthetic construct
  source 1..19: mol_type = other DNA
agatgtgtataagagacag

SEQ ID NO: 2 - DNA, 33 bp, synthetic construct
  source 1..33: mol_type = other DNA
tcgtcggcagcgtcagatgtgtataagagacag

SEQ ID NO: 3 - DNA, 19 bp, synthetic construct
  source 1..19: mol_type = other DNA
  modified_base 1: mod_base = OTHER; note = 2'-Deoxycytidine-5'-monophosphate
ctgtctcttatacacatct

SEQ ID NO: 4 - DNA, 34 bp, synthetic construct
  source 1..34: mol_type = other DNA
gtctcgtgggctcggagatgtgtataagagacag

SEQ ID NO: 5 - DNA, 35 bp, synthetic construct
  source 1..35: mol_type = other DNA
gcctccctcgcgccatagatgtgtataagagacag

SEQ ID NO: 6 - DNA, 36 bp, synthetic construct
  source 1..36: mol_type = other DNA
cttgccagcccgctcagagatgtgtataagagacag

SEQ ID NO: 7 - DNA, 53 bp, synthetic construct
  source 1..53: mol_type = other DNA
aatgatacggcgaccaccgagatctacacnnnnnnnngcctccctcgcgccat

SEQ ID NO: 8 - DNA, 51 bp, synthetic construct
  source 1..51: mol_type = other DNA
aatgatacggcgaccaccgagatctacacnnnnnnnntcgtcggcagcgtc

SEQ ID NO: 9 - DNA, 49 bp, synthetic construct
  source 1..49: mol_type = other DNA
caagcagaagacggcatacgagatnnnnnnnncttgccagcccgctcag

SEQ ID NO: 10 - DNA, 41 bp, synthetic construct
  source 1..41: mol_type = other DNA
ctgagtcggagacacgcannnnnnnngtctcgtgggctcgg

SEQ ID NO: 11 - DNA, 29 bp, synthetic construct
  source 1..29: mol_type = other DNA
aatgatacggcgaccaccgagatctacac

SEQ ID NO: 12 - DNA, 50 bp, synthetic construct
  source 1..50: mol_type = other DNA
caagcagaagacggcatacgagatnnnnnnnnctgagtcggagacacgca

SEQ ID NO: 13 - DNA, 24 bp, synthetic construct
  source 1..24: mol_type = other DNA
caagcagaagacggcatacgagat